SEQUENCE LISTING (82 sequences)

SEQ ID NO: 1 - DNA, 32 bp, Artificial Sequence
  Description of Artificial Sequence Synthetic oligonucleotide
ataagcttgaattctggcgttcatctccgacg

SEQ ID NO: 2 - DNA, 30 bp, Artificial Sequence
  Description of Artificial Sequence Synthetic oligonucleotide
ttcccgggtggatgctcagatgctagcgtc

SEQ ID NO: 3 - DNA, 35 bp, Artificial Sequence
  Description of Artificial Sequence Synthetic oligonucleotide
ggggtaccgacaacaatgcgtctatcttttgtgct

SEQ ID NO: 4 - DNA, 28 bp, Artificial Sequence
  Description of Artificial Sequence Synthetic oligonucleotide
ggggtacctcagctctgataccggtgaa

SEQ ID NO: 5 - DNA, 62 bp, Artificial Sequence
  Description of Artificial Sequence Synthetic oligonucleotide
atcgcactcgagctttcgcagatcccggggggcaatgagatatgactgagtactccgacgaa

SEQ ID NO: 6 - DNA, 63 bp, Artificial Sequence
  Description of Artificial Sequence Synthetic oligonucleotide
atcgcactcgagcttgtcgatcgacagatccggtcggcatctacttcagctctgataccggtg

SEQ ID NO: 7 - DNA, 59 bp, Artificial Sequence
  Description of Artificial Sequence Synthetic oligonucleotide
atcgcactcgagctttcgcagatcccggggggcaatgagatatgcgtctatcttttgtg

SEQ ID NO: 8 - DNA, 40 bp, Artificial Sequence
  Description of Artificial Sequence Synthetic oligonucleotide
tcgtccgtgctgcagctgctaacggcgacattgccggtgg

SEQ ID NO: 9 - DNA, 40 bp, Artificial Sequence
  Description of Artificial Sequence Synthetic oligonucleotide
tcgccgttagcagctgcagcacggacgagatctggagatt

SEQ ID NO: 10 - DNA, 40 bp, Artificial Sequence
  Description of Artificial Sequence Synthetic oligonucleotide
ccggtggagctgcagctgctgctcatgaagaggacgatgc

SEQ ID NO: 11 - DNA, 40 bp, Artificial Sequence
  Description of Artificial Sequence Synthetic oligonucleotide
tcatgagcagcagctgcagctccaccggcaatgtcgccgt

SEQ ID NO: 12 - DNA, 65 bp, Artificial Sequence
  Description of Artificial Sequence Synthetic oligonucleotide
gctgcagctgctaacggcgacattgccggtggagctgcagctgctgctcatgaagaggacgatgc

SEQ ID NO: 13 - DNA, 65 bp, Artificial Sequence
  Description of Artificial Sequence Synthetic oligonucleotide
agcagctgcagctccaccggcaatgtcgccgttagcagctgcagcacggacgagatctggagatt

SEQ ID NO: 14 - DNA, 44 bp, Artificial Sequence
  Description of Artificial Sequence Synthetic oligonucleotide
gctgcagcagctgcgggggctgctaccttcagcgtgactgacct

SEQ ID NO: 15 - DNA, 44 bp, Artificial Sequence
  Description of Artificial Sequence Synthetic oligonucleotide
agcagcccccgcagctgctgcagcatgagctcgaagaaatcttc

SEQ ID NO: 16 - DNA, 33 bp, Artificial Sequence
  Description of Artificial Sequence Synthetic oligonucleotide
ttcccggggacaacaatgcgactccactacgtg

SEQ ID NO: 17 - DNA, 38 bp, Artificial Sequence
  Description of Artificial Sequence Synthetic oligonucleotide
gtcagtcacgctgaaggtatcgagaacgccatgcccat

SEQ ID NO: 18 - DNA, 35 bp, Artificial Sequence
  Description of Artificial Sequence Synthetic oligonucleotide
attcccggggacaacaatgggcctccacaagggct

SEQ ID NO: 19 - DNA, 36 bp, Artificial Sequence
  Description of Artificial Sequence Synthetic oligonucleotide
gtcagtcacgctgaaggttaggtggtgtagtccgac

SEQ ID NO: 20 - DNA, 20 bp, Artificial Sequence
  Description of Artificial Sequence Synthetic primer
ttctccttttcactctcacg

SEQ ID NO: 21 - DNA, 21 bp, Artificial Sequence
  Description of Artificial Sequence Synthetic primer
agacacaaaatctgcaacttc

SEQ ID NO: 22 - DNA, 20 bp, Artificial Sequence
  Description of Artificial Sequence Synthetic primer
accttcagcgtgactgacct

SEQ ID NO: 23 - DNA, 19 bp, Artificial Sequence
  Description of Artificial Sequence Synthetic primer
gcgattgccaaccagttct

SEQ ID NO: 24 - DNA, 18 bp, Artificial Sequence
  Description of Artificial Sequence Synthetic primer
ggcgagcgtatgaccaag

SEQ ID NO: 25 - DNA, 18 bp, Artificial Sequence
  Description of Artificial Sequence Synthetic primer
gaaccaccgatccagacc

SEQ ID NO: 26 - DNA, 62 bp, Artificial Sequence; identical to SEQ ID NO: 5
  Description of Artificial Sequence Synthetic primer

SEQ ID NO: 27 - DNA, 61 bp, Artificial Sequence
  Description of Artificial Sequence Synthetic primer
gaaactcgagcttgtcgatcgacagatccggtcggcaggtacctcagctctgataccggtg

SEQ ID NO: 28 - DNA, 40 bp, Artificial Sequence
  Description of Artificial Sequence Synthetic primer
agatcccggggggcaatgagatatggacgggtccggggag

SEQ ID NO: 29 - DNA, 29 bp, Artificial Sequence
  Description of Artificial Sequence Synthetic primer
ggcaggtacctcagcccatcttcttccag

SEQ ID NO: 30 - DNA, 24 bp, Artificial Sequence
  Description of Artificial Sequence Synthetic primer
tcccgaattcagtttagcttcatg

SEQ ID NO: 31 - DNA, 26 bp, Artificial Sequence
  Description of Artificial Sequence Synthetic primer
tagtaagcttcccgatctagtaacat

SEQ ID NO: 32 - DNA, 28 bp, Artificial Sequence
  Description of Artificial Sequence Synthetic primer
ggaggaattcgctggctggtggcaggat

SEQ ID NO: 33 - DNA, 23 bp, Artificial Sequence
  Description of Artificial Sequence Synthetic primer
gtattggctagagaagcttgcca

SEQ ID NO: 34 - DNA, 57 bp, Artificial Sequence
  Description of Artificial Sequence Synthetic primer
agaaactcgagcttgtcgatcgacagatccggtcggcaggtacctcagctctgatac

SEQ ID NO: 35 - protein, 100 aa, Phytophthora sojae
FSAATDADQATVSKLAAAEFDTLVDVLTTESKRSLRATVDDGEERYKQFKIEALKKGKWTDIFNKWKGNELSPAEVQNKLKNKKLSDDLKDAIFRNYKDW

SEQ ID NO: 36 - protein, 91 aa, Phytophthora sojae
LSTTNANQAKIIKGTSPGGHSPRLLRAYQPDDEGDSPEDRTLSKAQVTKILNKLGKDVTWDHVMRNPALFQRYQKKANKIIEKQKAAAKNA

SEQ ID NO: 37 - protein, 429 aa, Phytophthora infestans
AGQSAGDLKDAIKAKNPATITCDAKDLQLSLAKTADGAWLPDDDQAALDLEDGKVHEDIQALIDGEKMKATWTIEDVLTANNMTKRKGRAPKSRQIHVLVVVPEGAFGSASETSKMDQLVEKVDKMYEQTVLGKRKYVHSEVTSTQGRQLLNDLDIRVEFVRTVPFDAGEGSSVDPYEWKRVIIENGEEVVLTEEQQRKRYRRYVEHNIGAVLKEKQLCVIGVERGTNILTVKVPGREIELAGRTDLLILSDLVAMRPTEVQYLPGVKMLIEVKRDVKASNDFQALSELIALDLLVDDPVMALLTDLKGEWIFFWVAEKINSSARIHKAAINKPGEAFEVIRALLVQPPTAPADTDTTEIKLPCFQSPVKRLKLRKALPPIGEGGDNGGIRESIERYYDIASMLGPDIEMARAVARQVTRSIPTFSYFS

SEQ ID NO: 38 - protein, 10 aa, Artificial Sequence
  Description of Artificial Sequence Synthetic peptide
GAPKKKRKVK

SEQ ID NO: 39 - protein, 14 aa, Artificial Sequence
  Description of Artificial Sequence Synthetic peptide
NELALKLAGLDINK

SEQ ID NO: 40 - protein, 14 aa, Artificial Sequence
  Description of Artificial Sequence Synthetic peptide
NELALKAAGADANK

SEQ ID NO: 41 - protein, 46 aa, Puccinia striiformis
TLLNVTRRLQNDGKPPDYCVDKWDEMMKERNKRLTGKPRGQCVDEI

SEQ ID NO: 42 - protein, 46 aa, Magnaporthe grisea
GISTVRWYTNGGKRPRRSIDQWDRQMMDRDRRLTGFLRGQSDNPIA

SEQ ID NO: 43 - protein, 43 aa, Melampsora lini
MEHVPAELTRVSEGYTRFYRSPTASVILSGLVKVKWDNEQMTM

SEQ ID NO: 44 - protein, 59 aa, Melampsora lini
HPMNSAKLAEEVKDGVNQPEFDRGFLRPFGAKMKFLKPDQVQKLSTDDLITYMAEKDKN

SEQ ID NO: 45 - protein, 33 aa, Melampsora lini
QYVVDPGFGEIECMCGQIARLTQRPFDVECEAT

SEQ ID NO: 46 - protein, 40 aa, Phytophthora sojae
TEYSDETNIAMVESPDLVRRSLRNGDIAGGRFLRAHEEDD

SEQ ID NO: 47 - protein, 66 aa, Phytophthora sojae
MRLSFVLSLVVAIGYVVTCNATEYSDETNIAMVESPDLVRRSLRNGDIAGGRFLRAHEEDDAGERT

SEQ ID NO: 48 - protein, 66 aa, Phytophthora sojae
MRLSFVLSLVVAIGYVVTCNATEYSDETNIAMVESPDLVRAAAANGDIAGGRFLRAHEEDDAGERT

SEQ ID NO: 49 - protein, 66 aa, Phytophthora sojae
MRLSFVLSLVVAIGYVVTCNATEYSDETNIAMVESPDLVRRSLRNGDIAGGAAAAAHEEDDAGERT

SEQ ID NO: 50 - protein, 66 aa, Phytophthora sojae
MRLSFVLSLVVAIGYVVTCNATEYSDETNIAMVESPDLVRAAAANGDIAGGAAAAAHEEDDAGERT

SEQ ID NO: 51 - protein, 66 aa, Phytophthora sojae
MRLSFVLSLVVAIGYVVTCNATEYSDETNIAMVESPDLVRRSLRNGDIAGGRFLRAHAAAAAGAAT

SEQ ID NO: 52 - protein, 14 aa, Phytophthora sojae
RFLRAHEEDDAGER

SEQ ID NO: 53 - protein, 7 aa, Plasmodium falciparum
RLLHETQ

SEQ ID NO: 54 - protein, 16 aa, Phytophthora infestans
RLLRKNEENEETSEER

SEQ ID NO: 55 - protein, 69 aa, Phytophthora sojae
MRLSFVLSLVVAIGYVVTCNATEYSDETNIAMVESPDLVRRSLRNGDIAGGRFLRAHEEDDAGERTFSV

SEQ ID NO: 56 - protein, 74 aa, Phytophthora sojae
MRLSFVLSLVVAIGYVVTCNATEYSDETNIAMVESPDLVRRSLRNGDIAGGRRRRRRRRRAHEEDDAGERTFSV

SEQ ID NO: 57 - protein, 76 aa, Phytophthora sojae
MRLSFVLSLVVAIGYVVTCNATEYSDETNIAMVESPDLVRRSLRNGDIAGGYGRKKRRQRRRAHEEDDAGERTFSV

SEQ ID NO: 58 - protein, 63 aa, Phytophthora sojae
MRLSFVLSLVVAIGYVVTCNADKYEKAVDYGFRESRILAEGEDTCARKEKTTLRKSKQKTFSV

SEQ ID NO: 59 - protein, 64 aa, Phytophthora sojae
MRLSFVLSLVVAIGYVVTCNAFNNNLCSKNAKGLNLNKRLLHETQAHVDDAAHHAHHVADTFSV

SEQ ID NO: 60 - protein, 66 aa, Phytophthora sojae
MRLSFVLSLVVAIGYVVTCNASYNKINSSSTYTHSRILKQLEFITLEEKTVNALQEMLDDSVTFSV

SEQ ID NO: 61 - protein, 68 aa, Phytophthora sojae
DLWNKVAAKKLAKAMLADPSKEQKAYEKWAKKGYSLDKIKNWLAIADPKQKGKYDRIYNGYTFHLYQS

SEQ ID NO: 62 - protein, 68 aa, Phytophthora sojae
DLWNKVGAKTLAKAMLKDPSKEQEAYKMWARKKYTLQDIKDWLAIADPKQKGKYDRIYDGYTFHLYQS

SEQ ID NO: 63 - protein, 68 aa, Phytophthora sojae
DLWNQVAAQQLAQAMLADPSKEQKAYEKWAKKGYSLDKIKNWLAIADPKQKGKYDRIYNGYTFHLYQS

SEQ ID NO: 64 - protein, 68 aa, Phytophthora sojae
DLWNKVAAKKLAKAMLADPSKEQKAAEKAAKKAYSGDKGKNALAIADPKQKGKYDRIYNGYTFHLYQS

SEQ ID NO: 65 - protein, 68 aa, Phytophthora sojae
DLWNKVAAKKLAKAMLADPSKEQKAYSKWASKGYSLSSIKSWLAIADPKQKGKYDRIYNGYTFHLYQS

SEQ ID NO: 66 - protein, 68 aa, Phytophthora sojae
DLWNKVAAKKLAKAMLADPSKEQKAYEKWAKKGYSLGGGGNWLAIADPKQKGKYDRIYNGYTFHLYQS

SEQ ID NO: 67 - protein, 68 aa, Phytophthora sojae
DLWNKVAAKKLAKAMLADPSKEQKAYEKWAKKGYSLDKGKNWLAIADPKQKGKYDRIYNGYTFHLYQS

SEQ ID NO: 68 - protein, 68 aa, Phytophthora sojae
DLWNKVAAKKLAKAMLADPSKEQKAYEKWAKKGYSLDKAKNWLAIADPKQKGKYDRIYNGYTFHLYQS

SEQ ID NO: 69 - protein, 68 aa, Phytophthora sojae
DLWNKVAAKKLAKAMLADPSKEQKAYEKWAKKGYSLDKVKNWLAIADPKQKGKYDRIYNGYTFHLYQS

SEQ ID NO: 70 - protein, 68 aa, Phytophthora sojae
DLWNKVAAKKLAKAMLADPSKEQKAYEKWAKKGYSLDKIKNWLAIADPKQKGAAAAIYAAYTAALYQS

SEQ ID NO: 71 - DNA, 20 bp, Artificial Sequence
  Description of Artificial Sequence Synthetic primer
tttttttttttttttttttt

SEQ ID NO: 72 - protein, 4 aa, Artificial Sequence
  Description of Artificial Sequence Synthetic peptide
AAAA

SEQ ID NO: 73 - protein, 9 aa, Artificial Sequence
  Description of Artificial Sequence Synthetic peptide
RRRRRRRRR

SEQ ID NO: 74 - protein, 4 aa, Phytophthora sojae
RFLR

SEQ ID NO: 75 - protein, 4 aa, Artificial Sequence
  Description of Artificial Sequence Synthetic peptide
KFLR

SEQ ID NO: 76 - protein, 4 aa, Artificial Sequence
  Description of Artificial Sequence Synthetic peptide
QFLR

SEQ ID NO: 77 - protein, 4 aa, Artificial Sequence
  Description of Artificial Sequence Synthetic peptide
FRLR

SEQ ID NO: 78 - protein, 4 aa, Artificial Sequence
  Description of Artificial Sequence Synthetic peptide
RFLQ

SEQ ID NO: 79 - protein, 4 aa, Artificial Sequence
  Description of Artificial Sequence Synthetic peptide
QFLQ

SEQ ID NO: 80 - protein, 4 aa, Artificial Sequence
  Description of Artificial Sequence Synthetic peptide
RFAR

SEQ ID NO: 81 - protein, 4 aa, Artificial Sequence
  Description of Artificial Sequence Synthetic peptide
RFVR

SEQ ID NO: 82 - protein, 4 aa, Artificial Sequence
  Description of Artificial Sequence Synthetic peptide
RFRL

(19) United States
(12) Patent Application Publication
(10) Pub. No.: US 2009/0011510 A1
(43) Pub. Date: Jan. 8, 2009

(54) Device and method for biolistic transformation of cells

(76) Inventors: Shiv D. Kale, Fairfax, VA (US); Brett M. Tyler, Blacksburg, VA (US)

Correspondence Address: LATIMER, MAYBERRY & MATTHEWS IP LAW, LLP, 13873 PARK CENTER ROAD, SUITE 106, HERNDON, VA, 20171, US

(21) Appl. No.: 12/134,906

(22) Filed: Jun. 6, 2008

Related U.S. Application Data
(60) Provisional application No. 60/942,474, filed on Jun. 7, 2007.

(51) Int. Cl. C12N 15/87; C12M 1/00

(52) U.S. Cl. 435/468; 435/285.3

(57) ABSTRACT

The present invention provides a device and method for biolistic bombardment of cells. In a preferred embodiment, the device comprises a hollow tube or cylinder that attaches to a standard biolistic transformation apparatus, and focuses the nucleic acids at cells and tissue sections. In a typical configuration, the device has two barrels that deliver equal or substantially equal amounts of nucleic acids to cells of a tissue, such as a plant leaf.

### CROSS-REFERENCE TO RELATED APPLICATIONS

This application relies on the disclosure and claims the benefit of the filing date of U.S. provisional patent application No. 60/942,474, filed on 7 Jun. 2007, the entire disclosure of which is hereby incorporated herein by reference.

### BACKGROUND OF THE INVENTION

1. Field of the Invention

The present invention relates to the field of molecular biology. More specifically, the invention relates to devices and methods for inserting exogenous nucleic acids into cells.

2. Description of Related Art

Biolistic transformation is a technique that is used extensively to introduce nucleic acids, and especially DNA, into various cells. It is used to create both stably and transiently transformed cells containing exogenous nucleic acids. It is widely applicable to cell types, and has been used to transform both prokaryotes and eukaryotes. For example, there are numerous reports of use of biolistic transformation of plant cells, animal cells, fungal cells, and bacterial cells to create both stably transformed cells and transiently transformed cells.

Biolistic transformation can be used in the context of any number of scientific goals. Thus, it can be used to create transformed cells for research on the role of a particular gene in a biochemical or disease process. Likewise, it can be used to create recombinant cells for production of a particular nucleic acid or protein of interest, such as for research use or therapeutic use. Indeed, the use of biolistic transformation with appropriate devices (commonly referred to as “gene guns”) is widespread and common in the field, particularly for production of recombinant or transformed cells for research purposes.

Generally speaking, genetic transformation by biolistic particle bombardment is a technique by which DNA is delivered into living cells by bombarding the tissues with tungsten or gold particles coated with a DNA preparation (Klein et al., 1987). Early versions of the technique used shotgun cartridges to propel the particles, but modem devices use a blast of helium gas (Sanford et al., 1991). Biolistic transformation has been used to generate stably genetically modified organisms ranging from animals (including humans) to fungi, bacteria, and plants (Klein et al., 1992). Biolistic transformation has also been used for transient expression of genes in living cells (Klein et al., 1992). The purpose of transient expression is typically to determine the function of the gene or genes delivered into the cells, rather than to create permanently modified cells. The biochemical activity of the gene product can be assayed directly, or more commonly a reporter gene encoding beta-glucuronidase (GUS) is fused to the gene to monitor gene expression (Klein et al., 1992). Another useful version of the transient expression assay for gene products that kill cells is to bombard the cells of interest with DNA containing both the GUS gene and the gene of interest and to measure the reduction in transformed cells caused by cell killing in reference to a control (Mindrinos et al., 1994).

A major limitation of quantitative transient biolistic transformation is the significant variability, both from shot to shot and among replicate samples of the targeted tissue, for example different leaves of a plant (e.g., Schledzewski and Mendel, 1994). This limitation results in the requirement for very large numbers of shots to be performed to obtain statistical significance, and the inability to measure small differences between treatment and control.

The inventors have realized that there thus exists a need in the art for improved devices and methods for transforming cells by way of biolistic transformation. The improved methods and devices should preferably improve (i.e., reduce) variability in the amounts of nucleic acids that are introduced into a cells from one bombardment attempt to another. The improved devices and methods should accordingly improve sensitivity and reproducibility of transformations using this technology.

### SUMMARY OF THE INVENTION

The present invention provides improved devices and methods for biolistic transformation of cells. In general, the invention provides a “gene gun” that provides highly-reproducible results for introduction of biological or chemical material into a cell. In a preferred embodiment, the invention provides a biolistic gun that introduces nucleic acids into a cell. Accordingly, it provides for methods of reproducibly introducing a nucleic acid into a target cell to create a recombinant or transformed cell. In some embodiments, the device is a helium-driven biolistic gun that enables very precise comparisons of treatment and control, or any two treatments. Typically, the device is a device that is based on a biolistic gun available in the art, which is modified to provide improved results, as discussed below. In embodiments, the modification is to include two barrels, rather than a single barrel, to the gene gun so that two blasts of particles, loaded with different DNA preparations may be fired side-by-side into the same tissue samples, such as a leaf.

Thus, in embodiments, the invention is a modification of existing technology, where the device of the invention comprises an additional part that fits an existing apparatus for particle bombardment, such as one that introduces transient expression of a reporter or other functional gene (e.g., a device available from Bio-Rad). The modification provides two or more barrels for simultaneous or staggered/sequential application/penetration of genetic material into living cells, which can be compared to the Bio-Rad device, which does not include a barrel, but rather provides a single aperture, through which the nucleic acid material is expelled. The device and method of the invention are particularly well suited for plants, and provide an advantage over current biolistic gun devices and methods. In addition, the device and method of the invention provide a good alternative to Agrobacterium-mediated transfer of genetic material into plant cells, which is severely limited by, among other things, gene size, need to clone into a vector, and variability among species. Furthermore, the invention provides, in embodiments, the ability to introduce nucleic acids into any thin layer or mono-layer of cells, including but not limited to in vitro cultures of a wide variety of cells or unicellular adherent organisms. While gene guns have been on the market for 15-20 years, precise replication of controls or dose-response comparisons has been a big problem, with internal controls being the only solution currently. An exemplary advantage of this invention is that it allows a range of new applications that are enabled by the improved accuracy and precision inherent in this device and methods. For example, the presently disclosed apparatus allows quantitative comparisons for dose-response titers of gene expression, or for a series of mutations to a promoter or a gene of interest. The device allows nucleic acids, for example, to enter a cell in a context (comparison of test and control) that results in very accurate and precise measurements of the functions of the proteins encoded by the genes carried on the nucleic acid.

### BRIEF DESCRIPTION OF THE DRAWINGS

The accompanying drawings, which are incorporated in and constitute a part of this specification, illustrate an embodiment of the device of the invention, and together with the written description, serve to explain certain principles of the invention.

FIG. 1 depicts an attachment for a biolistic gun, which can attach to a standard biolistic gun and convert the biolistic gun to a two-barrel delivery device. Panel A depicts an isometric view; Panel B depicts a front view; Panel C depicts a top view; and Panel D depicts a bottom view.

FIG. 2 depicts the attachment of FIG. 1, when attached to a biolistic gun available in the art. Panel A shows one embodiment, termed the “production embodiment” and Panel B depicts another embodiment, termed the “original embodiment”.

FIG. 3 shows experiments that confirm a finding made with double barreled gene gun, namely that the RXLR and dEER motifs are required for Avr1b function in Phytophthora sojae (P. sojae) transformants produced by protoplast fusion. Panel A depicts the sequences of the mutations in the RXLR1, RXLR2, and dEER motifs. Panel B shows Pst I restriction enzyme analysis of PCR products. Panel C depicts the detection of Avr1b mRNA in P. sojae stable transformants by RT-PCR. Panel D shows the distributions of HMM scores of RXLR flanking regions for non-permuted, permuted, and curated proteins. Panel E depicts the phenotype of transformants carrying the indicated wild type or mutant Avr1b-1 genes.

FIG. 4 depicts a common host targeting mechanism in oomycetes and Plasmodium. Panel A shows the features and functional exchange of host targeting signals in Ph. sojae Avr1b, Pl. falciparum HRPII and P. infestans Avr3a. Panel B depicts the anatomical contexts of oomycete and Plasmodium effector entry.

FIG. 5 depicts the use of the device of the invention to demonstrate RXLR and dEER functions. Panel A shows the ratio of blue spots in the presence of various forms of Avr1b-1 compared to the control. Panels B and C depict a direct comparison of bombardment with mature Avr1b and secretory Avr1b, respectively.

FIG. 6 shows the reproducibility between the two barrels of one embodiment of the device. Data is from Table 5.

FIG. 7 shows the use of the device of the invention in comparing DNA constructs comprising the Avr1b gene or the GUS gene in a cell-killing assay. Panel A depicts a schematic of the DNA constructs. Panel B shows a schematic of the cell-killing assay. Panel C shows the results of the cell-killing assay.

FIG. 8 depicts the use of the device of the invention to produce precise dose-response curves as seen by the cell-killing assay causes by three different toxins.

FIG. 9 shows the use of the device to assay the functional replacement of Avr1b transport signal with protein transduction motifs and Plasmodium host targeting signals. Panel A depicts the sequences of modified Avr1b proteins. Panel B shows the results of the assays as determined by the ratio of blue spots in the presence of Avr1b-1 compared to the control.

FIG. 10 depicts the mutants of Avr1b used for assay of PCD suppression in Table 13.

### DETAILED DESCRIPTION OF VARIOUS EMBODIMENTS OF THE INVENTION

Reference will now be made in detail to an exemplary embodiment of the invention, an example of which is illustrated in the accompanying drawings. The following detailed description is provided to assist the reader in understanding certain features and embodiments of the invention, and should not be understood as limiting the invention to any particular embodiment or combination of elements, or to any particular method steps or order of steps.

In one aspect, the invention provides a device (also referred to herein at various times as an “attachment”) for introducing biological or chemical material into intact cells or tissue. Although the material is typically nucleic acids, it is envisioned that other forms of biological or chemical material can be used in the device, such as proteins, virions, chemical drugs, vaccines, immunogens for cancer immunotherapy, and the like.

In a preferred embodiment, the device of the invention introduces nucleic acids into a cell and is comprised of a means for attaching the device to a biolistic transformation apparatus known in the art, as well as a means for focusing the nucleic acid to a cell or collection of cells. The means for attaching the device may comprise a retaining spring or screw thread, for example, which holds the attachment in an appropriate position in a biolistic gun that is commercially available. The attachment means may be altered in any suitable way to achieve attachment of the device of the invention to the biolistic gun. The invention is not limited by any one way of attaching the device to a biolistic transformation apparatus as there are many ways to connect the device.

In one embodiment, the device of the invention is a device for introducing one or more payloads into a biological cell of a biological tissue, said device comprising an apparatus capable of ejecting said payload into said cell, and an attachment for focusing said payload on a specific location of said tissue, wherein said attachment comprises at least two distinct chambers for transmission of said payload(s) from said apparatus to said cell, and wherein said attachment is connected to said apparatus such that an aperture or opening in said apparatus for ejection of said payload is aligned with said chambers such that ejection of said payload permits the payload to traverse the chambers and contact said cell. The payload may be a biological or chemical material. The attachment for focusing the payload may comprise two hollow cylinders or tubes and the attachment may be attached to the apparatus by way of a retaining spring or a screw thread. The device may be comprised of a metal or plastic, or a combination of both.

Stated another way, the device of the invention is an attachment for a biolistic transformation apparatus, said attachment comprising a connector for connecting the attachment to the apparatus, and at least two chambers for delivery of a payload from the apparatus to a target. The attachment may comprise two chambers and the connector may comprise a spring clip or screw thread.

The means for focusing the nucleic acids may comprise at least two hollow cylinders or tubes, but may also comprise other three dimensional shapes that allow for focusing the nucleic acids. For example, it may take the form of a cone, pyramid, or other shape that has a different cross-sectional dimension at its proximal and distal ends. Where the focusing means has a different cross-sectional dimension at one end as compared to the other, either the smaller or the larger end may be proximal to the biolistic transformation apparatus to which it is attached.

The nucleic acids that can be introduced into cells include polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including a basic sites). Thus, “nucleic acid” includes double- and single-stranded DNA, as well as double- and single-stranded RNA. The term “nucleic acid”, as used herein, also includes polymers of ribonucleosides or deoxyribonucleosides that are covalently bonded, typically by phosphodiester linkages between subunits, but in some cases by phosphorothioates, methylphosphonates, and the like. Such nucleic acids include, but are not limited to, gDNA; hnRNA; mRNA; noncoding RNA (ncRNA), including but not limited to rRNA, tRNA, miRNA (micro RNA), siRNA (small interfering RNA), snORNA (small nucleolar RNA), snRNA (small nuclear RNA), and stRNA (small temporal RNA); fragmented nucleic acid; nucleic acid obtained from subcellular organelles, such as mitochondria or chloroplasts; and nucleic acid obtained from microorganisms, parasites, or DNA or RNA viruses that might be present in a biological sample. Synthetic nucleic acid sequences, that might or might not include nucleotide analogs, are also within the scope of the invention. The size of the nucleic acids (and therefore the size of the proteins expressed by this device and corresponding method) can vary from very small (such as 50 bp) to large (such as up to 500,000 bp). A person with ordinary skill in the art will know what sizes of nucleic acids are appropriate for particle bombardment and can vary the sizes according to the goal of the method.

The nucleic acids are typically attached to gold or tungsten particles before placement into the biolistic gun. Of course, other materials (e.g. silicon) are contemplated to work in the invention, as long as the nucleic acids can adhere to the material and can be released after breaching the cell membrane. The material, although preferably in the shape of ball (or otherwise substantially spherical), can also be used in another shape, such as a cube, an elongated cube, a cylinder, and the like. In some biolistic guns, a material delivery system, such as gold or tungsten balls, may not even be required.

The invention is not limited in the kinds of intact cells or tissues that can be used for bombardment. The device is applicable to bacterial cells and animal tissue or cells, such as but not limited to, humans, primates, canines, felines, bovines, ovines, porcines, equines, and rodents. However, the device is envisioned to be used primarily for plant or algal tissues or cells. This includes cereals and other monocotyledons, as well as dicotyledons. Some examples of plants include, but are not limited to, tomato, rice, cotton, tobacco, corn, grasses, and soybean. The device can be applied to any part of a plant, such as but not limited to, the stem, the leaf, the seed, the roots, and the flowers. The biological or chemical material can be delivered by bombardment into various parts of the cell, such as but not limited to, the nucleus, the cytoplasm, the mitochondria, and the chloroplasts. If the biological material is a nucleic acid, the biological material may incorporate itself into the nuclear, mitochondrial, or chloroplast genome to permanently transform the cell. In other cases, particle bombardment may allow transient expression to occur where the nucleic acid does not permanently become a part of the cellular genome.

In general, the device comprises one or more hollow cylinders, tubes, barrels, etc. that direct nucleic acid material from a biolistic gun to at least one target cell. The device may be comprised of one or more metals (e.g., iron, steel, aluminum, copper), wood, plastic, or any other material that will allow the apparatus to function. In preferred embodiments, the device is primarily made of a metal or a kind of plastic, or a combination of the two. In embodiments, the device is a modification of existing technology, where the device comprises an additional part that fits an existing apparatus for particle bombardment. In other embodiments, the device is manufactured as a single piece with more than one barrel, such as two barrels, three barrels etc. The gas used for particle bombardment is typically helium, but may also be nitrogen or air, or any other gas or combination of gasses that will allow the nucleic acids to be delivered to the sample tissue.

The device of the invention is typically used for comparing a treatment and a control, or two treatments. Because the blasts of particles that are fired into tissue samples can be fired side-by-side, the blasts can be compared with precision. This kind of use of the device is especially helpful in biological research, where controls are needed. However, the device may also be directed to other applications in both plants and animals. In some embodiments in plants, the device can be used to produce pharmaceutical drugs in plants, plants that are resistant to weeds or insects, or to produce high-yield plants. Other applications for inserting exogenous nucleic acids into plant cells will immediately be apparent to those of skill in the art. Applications in animals or humans include, but are not limited to, genetic vaccination where genes are introduced into a subject to elicit an immune response to the proteins expressed by the delivered gene, suicide gene therapy where a gene that expresses a toxic protein but has tumor specific promoters is introduced to tumor cells, genetic pharmacology where the device of the invention can be used to introduce genes that will produce proteins that are useful or therapeutic to an organism, and the like. Other applications will be apparent to those of skill in the art.

In another aspect, the invention provides a method for inserting or introducing biological or chemical material into a cell. In a preferred embodiment, the method is used for inserting nucleic acid into a cell. In general, the method comprises causing the nucleic acid to penetrate the cell exterior surface and remain inside the cell using a device or attachment according to the present invention. The cell may be any cell, including prokaryotic and eukaryotic. Thus, the cell may be a bacterial cell, a plant cell, or an animal cell, for example. Typically, the nucleic acid is propelled through the cell's surface, such as through the cell wall or cell membrane, by way of force provided to the nucleic acid from a device to which the device or attachment of the invention is attached. In preferred embodiments, the method comprises introducing the nucleic acid by way of two or more independent barrels to two or more distinct regions of a tissue comprising cells intended for nucleic acid insertion (“target cells”). Stated another way, in a preferred embodiment, the steps of the method include introducing a nucleic acid into a cell by contacting the cell with the nucleic acid with sufficient force to cause the nucleic acid to enter the interior of the cell to create a recombinant or transformed cell, wherein the force propelling the nucleic acid is provided by an apparatus comprising the device of the invention. Preferably, the contacting of the cell by the nucleic acid does not kill the cell and preferably, the nucleic acid is DNA. Further, the method may comprise exposing the recombinant or transformed cell to conditions that allow the cell to live and, optionally, grow and/or reproduce. Stated another way, the method of the invention is a method of introducing a payload into a biological cell, said method comprising contacting the cell with the payload with sufficient force to cause the payload to enter the interior of the cell, wherein the force propelling the payload is provided by the device of the invention. Preferably, the contacting in the method does not kill the cell. The payload may be a biological or chemical material, such as a nucleic acid. In some embodiments, the nucleic acid is DNA and the cell is rendered recombinant or transformed with the DNA. The method may also comprise exposing the recombinant or transformed cell to conditions that allow the cell to live and, optionally, reproduce. In another embodiment, the method is a method of introducing a nucleic acid into a biological cell, said method comprising contacting said cell with a nucleic acid emitted from an apparatus with sufficient force to cause the nucleic acid to enter the cell, wherein said apparatus comprises a biolistic transformation apparatus comprising an attachment comprising two or more chambers that focus the nucleic acid onto specific cells of a tissue. The method may produce a transformed plant cell, which may stably maintain the introduced nucleic acid.

Among the many features and/or advantages of the present invention is the fact that each barrel of the attachment of the invention is capable of providing identical, substantially identical, or insignificantly different results from the other barrel(s), when considered from the context of insertion of a nucleic acid of interest and/or expression of an inserted nucleotide sequence within a target cell or tissue. This eliminates at least one variable in gene expression comparisons of two different nucleic acid constructs in plants, where leaf to leaf variation can result in a significant difference in gene expression. The ability to bombard two constructs into one leaf at the same time helps to significantly reduce the variability in gene expression comparison experiments.

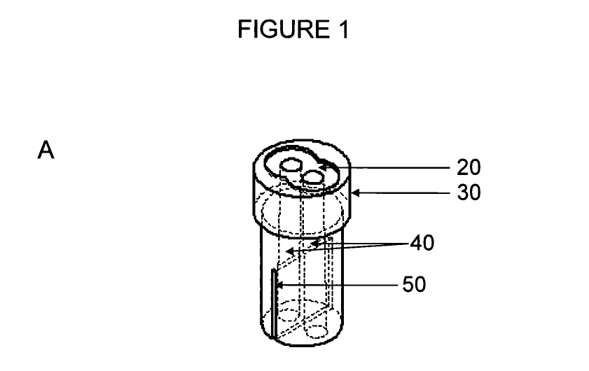

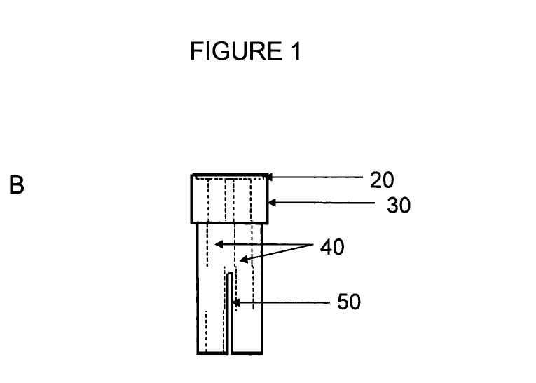

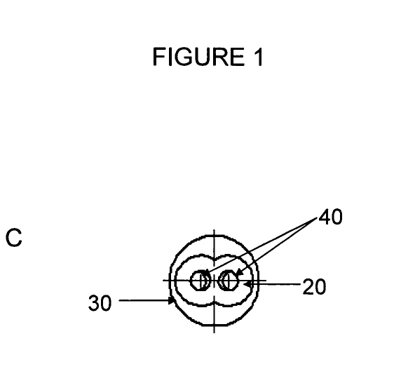

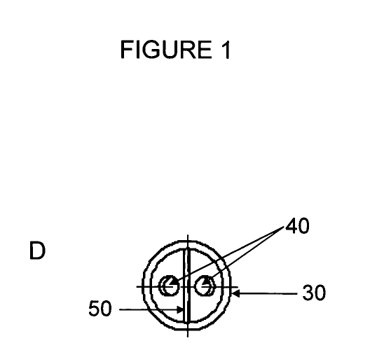

An embodiment of the attachment according to the invention is depicted in FIG. 1, which depicts a double barrel attachment. The attachment was designed to, and can be used to, replace an internal ring inside of the microcarrier launch assembly of a Biolistic Particle Delivery System-1000/He available from Bio-Rad Laboratories, Inc. (Hercules, Calif.). In this Figure, Panel A depicts an isometric view, Panel B depicts a front view, Panel C depicts a top view and Panel D depicts a bottom view of an embodiment of the device. In Panel A, it can be seen that the attachment 10 comprises a dual stopping screen support (ring/platform) 20, a nest 30, two barrels 40, a slit 50 for a separator 60 (not depicted). A separator 60 extends from the split in the barrels to the specimen for shooting. This separator 60 reduces or prevents accidental spread of particles from one barrel's target region into the other barrel's target region. In Panel B, the length of two barrels 50 can be seen. Panels C and D depict a top view and a bottom view of the two barrels 50 of the device, respectively.

The typical size of the device of the invention is of a size that can easily fit onto a biolistic gun. The particle bombardment apparatus comprising a biolistic gun and the attachment of the invention are of a size that can be easily manipulated. In embodiments, the apparatus can be easily carried. A typical size for the device of the invention is a length of 2 to 3 inches. Of course, the device may range in size from 0.5 inches to several feet depending on the size of the biolistic gun to which it is attached. A preferred embodiment for the apparatus is approximately 1 inch in diameter so that it may fit into the existing fixed nest with retaining spring. A preferred embodiment for the barrels of the device is approximately 1 cm in diameter. The diameter of the barrel can vary anywhere from 0.1 inch to 1 foot.

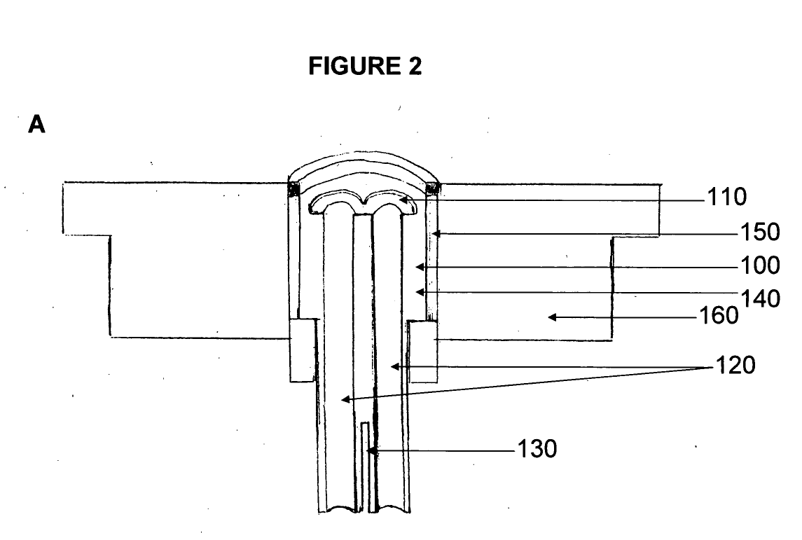

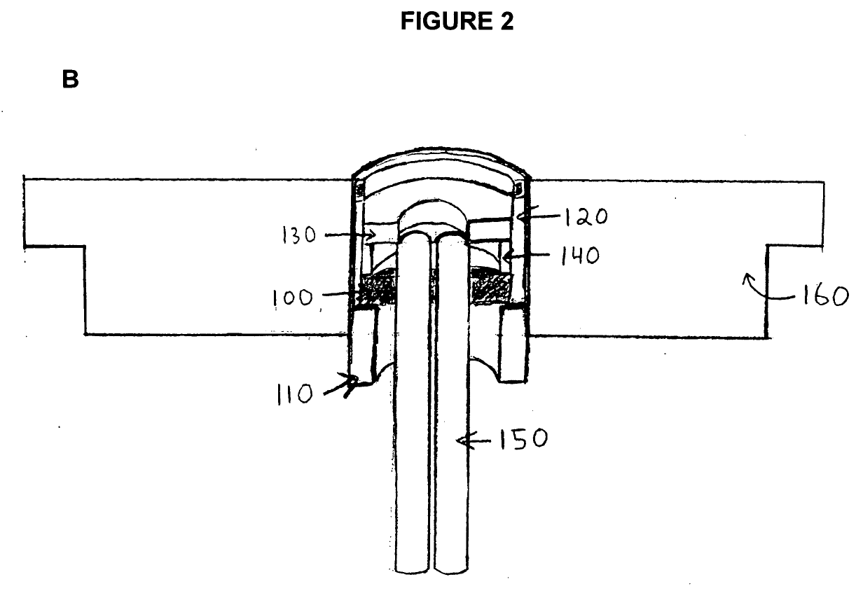

As can be seen from FIG. 2 (Panel A), the attachment can easily be connected to the commercially available product to become a part of the system. In this Figure, it can be seen that the attachment 100 comprises a dual stopping screen support (ring/platform) 110, a nest 140, two barrels 120, a slit 130 for a separator (not depicted). The attachment 100 fits into a fixed nest with a retaining spring 150 or another means for attaching and holding the attachment to a biolistic device. The fixed nest with a retaining spring 150 fits into a launch assembly shelf with a recessed set screw 160. Element 160 with all of its other assembled parts (100-150) fits inside a shelf in a typical gene gun apparatus. In this embodiment, the barrels 150 are locked into place upon assembly with the rest of the system; however, any other suitable attachment means may be used (e.g., screw threads, bolts/nuts, screws). Connection of the double barrel attachment allows for two targeted, simultaneous shots on a single specimen. In this configuration, the barrels 150 are placed directly under the stopping screen support of the commercially available product, reducing or preventing any possible errant particles of tungsten from entering the wrong barrel. The shots are individually loaded above their respective barrel. A metal/plastic separator extends from the split in the barrels to the specimen for shooting (not depicted). This separator reduces or prevents accidental spread of particles from one barrel's target region into the other barrel's target region.

It is envisioned that other modifications can be made to the device of the invention and/or to a biolistic transformation apparatus that it is attached to it. For example, in another embodiment, the apparatus would only replace a spacer ring inside the fixed nest retaining spring (200). Two barrels would be connected to the spacer ring and be directly under the stopping screen support ring (240). A separator would still be added between the barrels (250). This original embodiment (FIG. 2, Panel B) versus the production embodiment (FIG. 2, Panel A) differ in several ways. The production embodiment (Panel A) is easier to mass produce and clears the opening to the barrels, whereas in the original apparatus (Panel B), the barrel openings are slightly blocked. The production embodiment replaces the stopping screen support ring (230), a spacer ring (240). This allows a completely clear opening to the barrels and gives further separation between both tungsten preps. The production embodiment's sole purpose is to ease production. Less cutting and less complex molds are required. Both function identically and are based on the same concept. The embodiment of the apparatus has been physically manifested in two forms, one for developmental purposes and the other in preparation for mass production.

As should be evident, in addition to the attachment, the invention provides a biolistic transformation apparatus comprising it. In general, the apparatus comprises a biolistic transformation apparatus substantially or fully identical to one currently commercially available and an attachment as described above. The two elements are connected in any suitable fashion that allows for emission of material from the apparatus, through the attachment, and into or onto a target of interest, such as a plant cell. The combination apparatus functions to deliver a payload of interest to a target in a manner similar to a currently commercially available biolistic apparatus, but is capable of delivering two or more payload samples, which may be identical or different and delivered simultaneously or at different times.

The device and method of the invention can be modified in many ways within the context of the invention. For example, the double barrel addition can also be further modified to incorporate more barrels if desired. Doing so would likely require modifying the microcarrier assembly as well. Further, the current shape of the microcarrier assembly might prevent the full exposure of the opening of both barrels in some configurations. In those situations, the microcarrier assembly can be redesigned to allow for further clearing of the barrel openings. In addition, the disposable microcarrier can be redesigned in some embodiments to have two cupules that would align over the barrels and cradle this bombardment preparation. Doing so would help increase precision. Alternatively or additionally, the barrel length and diameter can be modified to suit the target specimen. We have shown that a certain length of barrel rupture pressure and vacuum pressure are ideal for a specific target specimen (soybean leaves). Other target samples might benefit from differently calibrated sizes for optimization.

Use of the attachment and combination apparatus described above to deliver a payload of interest to a target is contemplated. Included in this aspect is use to deliver two or more payloads, which can be the same or different and at the same or different amounts or concentrations, to the same or different cells or tissues of a target organism. Thus, use of the attachment to deliver biological molecules, such as DNA or RNA, or chemical substances, such as drugs, is contemplated.

### EXAMPLES

The following examples are provided to show certain features of the attachment of the invention and show effectiveness under certain conditions. The examples are not to be understood as a limitation in any way of the invention.

### Example 1

### Use of Device for Transforming Plant Cells and Discovery of Pathogen Host-Targeting Motif

Pathogens use effector molecules to manipulate the physiology of their hosts, to make them more susceptible to infection. Effector proteins secreted by oomycete and fungal pathogens have been inferred to enter host cells, but the mechanism of entry is unknown. We identify here a motif, RXLR, required for entry of the oomycete effector protein Avr1b into soybean host cells, and show that the motif functions in the absence of the pathogen. The confirmation of RXLR as a targeting motif indicates that the more than 350 RXLR containing genes found in two oomycete genome sequences encode potential effectors. The RXLR motif closely resembles the erythrocyte targeting signal of Plasmodium effector proteins suggesting that the machinery of the hosts (soybean and human) targeted by the pathogens is very ancient.

The following experiments were performed with P. sojae isolate P7076 (Race 19, Avr1b-) (Forster et al., 1994), which was routinely grown and maintained on V8 agar using standard techniques. The following protocol was used to transform P. sojae cells: three day-old P. sojae mycelial mats, cultured in pea broth medium, were rinsed and washed in 0.8M mannitol, then placed in enzyme solution [0.4M mannitol, 20 mM KCl, 20 mM 2-(N-morpholino) ethanesulfonate (MES) pH 5.7, 10 mM CaCl₂, 10 mg/ml β-1,3 glucanase (InterSpec 0439-1) and 5 mg/ml cellulysin (Calbiochem 219466)] and incubated for 40 minutes at 22° C. with 100 rpm shaking. The protoplasts were harvested by centrifugation at 1500 rpm for 3 min and resuspended in W5 solution (5 mM KCl, 125 mM CaCl₂, 154 mM NaCl, 31 mg/ml glucose) at a concentration of 2×10⁶ protoplasts/ml or higher. After 30 minutes, the protoplasts were centrifuged at 1500 rpm for 4 min and resuspended in an equal volume of MMg solution (0.4M mannitol, 15 mM MgCl₂ and 4 mM MES pH 5.7) to allow protoplasts swell. To each of 1 ml MMg solution, 25 ug transforming DNA was added and incubated for 10 minutes on ice. Then, three aliquots of 580 ul each of freshly made PEG solution (40% v/v polyethylene glycol 4000, 0.3 M mannitol, 0.15 M CaCl₂) were slowly pipetted into the protoplast suspension and gently mixed. After 20 minutes incubation on ice, 10 ml pea broth containing 0.5M mannitol were added and the protoplasts incubated overnight to regenerate. The regenerated protoplasts were suspended in liquid pea agar (40° C.) containing 0.5M mannitol and 50 ug/ml G418 and plated. The visible colonies could be observed after 2-3 days incubation at 22° C. All transformants were propagated on V8 agar with 50 ug/ml G418 at 22° C.

For plasmid construction, all plasmids were propagated in E. coli strains DH5 or DH10B using standard methods. Purification of plasmid DNA was carried out using the QIAprep Spin Miniprep Kit (QIAGEN, Cat. No. 27104) or the QIAGEN Plasmid Maxi Kit (QIAGEN, Cat. No. 12163) using provided protocols. All the plasmid constructs were confirmed with sequencing by the Virginia Bioinformatics Institute (VBI) Core Laboratory Facility.

The oligonucleotides and plasmids used in this study are described in Tables 1 and 2, respectively.

TABLE 1

| | | |
|---|---|---|
| Name | Applications | Sequence (from 5′ to 3′) |

| | | |
|---|---|---|
| UF | Hind III and Sma I sites | ataagcttgaatTCTGGCGTTCATCTCCGACG |
| | added to rpL41 promoter | (SEQ ID NO:1) |
| | to drive G418 resistance | |
| | gene | |
| UR | | ttcccgggTGGATGCTCAGATGctagcGTC |
| | | (SEQ ID NO:2) |
| PrimerC | Kpn I sites added | ggggtaccgacaacaATGCGTCTATCTTTTGTGCT |
| | flanking Avr1b for | (SEQ ID NO:3) |
| | insertion downstream of | |
| | HAM34 promoter | |
| PrimerD | | ggggtaccTCAGCTCTGATACCGGTGAA |
| | | (SEQ ID NO:4) |
| Avr1bF | Xho I site and initation | atcgcactcgagctttcgcagatcccggggggcaATGAGATATG |
| | codon added to 5′ end of | ACTGAGTACTCCGACGAA |
| | mature Avr1b for | (SEQ ID NO:5) |
| | insertion downstream of | |
| | CaMV 35S promoter | |
| Avr1bR | Xho I site to 3′ end of | atcgcactcgagcttgtcgatcgacagatccggtcggcatctactTCAG |
| | Avr1b for insertion | CTCTGATACCGGTG |
| | downstream of CaMV | (SEQ ID NO:6) |
| | 35S promoter | |
| Avr1bfull_F | Same as Avr1bF but for | atcgcactcgagctttcgcagatcccggggggcaATGAGATatgC |
| | secretory Avr1b | GTCTATCTTTTGTG |
| | | (SEQ ID NO:7) |
| Motif1F | Introduction of RxLR1 | TCGTCCGTgctgcagctgctAACGGCGACATTGCCG |
| | mutation, creating a Pst I | GTGG |
| | site | (SEQ ID NO:8) |
| Motif1R | | TCGCCGTTagcagctgcagcACGGACGAGATCTGG |
| | | AGATT |
| | | (SEQ ID NO:9) |
| Motif2F | Introduction of RxLR2 | CCGGTGGAgctgcagctgctGCTCATGAAGAGGAC |
| | mutation, creating a Pst I | GATGC |
| | site | (SEQ ID NO:10) |
| Motif2R | | TCATGAGCagcagctgcagcTCCACCGGCAATGTC |
| | | GCCGT |
| | | (SEQ ID NO:11) |
| Motif1 + 2F | Introduction of RxLR1 & | gctgcagctgctAACGGCGACATTGCCGGTGGAgctg |
| | RXLR2 mutations, | cagctgctGCTCATGAAGAGGACGATGC |
| | creating 2 Pst I sites | (SEQ ID NO:12) |
| Motif1 + 2R | | agcagctgcagcTCCACCGGCAATGTCGCCGTTagca |
| | | gctgcagcACGGACGAGATCTGGAGATT |
| | | (SEQ ID NO:13) |
| Motif3F | Introduction of dEER | gctgcagcagctGCGGGGgctgctACCTTCAGCGTGAC |
| | mutation, creating a Pst I | TGACCT |
| | site | (SEQ ID NO:14) |
| Motif3R | | agcagcCCCCGCagctgctgcagcATGAGCTCGAAGA |
| | | AATCTTC |
| | | (SEQ ID NO:15) |
| HpAvh341F | 5′ flanking Kpn I site | ttcccggggacaacaATGCGACTCCACTACGTG |
| | added to HpAvh341 for | (SEQ ID NO:16) |
| | insertion downstream of | |
| | HAM34 promoter | |
| HpAvh341R | For fusion with Avr1b | GTCAGTCACGCTGAAGGTATCGAGAACGCCA |
| | | TGCCCAT |
| | | (SEQ ID NO:17) |
| PsAvh171F | 5′ flanking Kpn I site | attcccggggacaacaATGGGCCTCCACAAGGGCT |
| | added to PsAvh171 for | (SEQ ID NO:18) |
| | insertion downstream of | |
| | HAM34 promoter | |
| PsAvh171R | For fusion with Avr1b | GTCAGTCACGCTGAAGGTTAGGTGGTGTAGT |
| | | CCGAC |
| | | (SEQ ID NO:19) |

TABLE 2

| Plasmid Name | Source | Construct | Construction Strategy |
|---|---|---|---|
| pUN | pHamNptII (S5) | NptII gene for G418 resistance | Ham34 promoter replaced by P. sojae |
| | | fused to P. sojae rpL41 promoter | rpL41 promoter using |
| | | (S6) | primers UF and UR |
| pHamAvr1b | pHamNptII | Avr1b-1 gene fused to Ham34 | NptII gene replaced with P. sojae |
| | | promoter for P. sojae | Avr1b-1 using PrimerC and |
| | | transformation | PrimerD |
| M1 | pHamNptII | Avr1b-1(RXLR1) mutant fused to | Same as pHamAvr1b with |
| | | Ham34 promoter | mutation introduced by primers |
| | | | motif1F and motif1R |
| M2 | pHamNptII | Avr1b-1(RXLR2) mutant fused to | Same as pHamAvr1b with |
| | | Ham34 promoter | mutation introduced by primers |
| | | | motif2F and motif2R |
| M1 + 2 | pHamNptII | Avr1b-1(RXLR1 + 2) mutant fused | Same as pHamAvr1b with |
| | | to Ham34 promoter | mutation introduced by primers |
| | | | motif1 + 2F and motif1 + 2R |
| M3 | pHamNptII | Avr1b-1(dEER) mutant fused to | Same as pHamAvr1b with |
| | | Ham34 promoter | mutation introduced by primers |
| | | | motif3F and motif3R |
| pHamAvh341 | pHamNptII | HpAvh341 fused with C-terminal | NptII gene replaced with |
| | | domain of Avr1b, driven by | HpAvh341-Avr1bfusion using |
| | | Ham34 promoter | primers HpAvh341F, |
| | | | HpAvh341R and PrimerD |
| pHamAvh171 | pHamNptII | PsAvh171 fused with C-terminal | NptII gene replaced with |
| | | of Avr1b drived by Ham34 | PsAvh171-Avr1b fusion using |
| | | promoter | primers PsAvh171F, PsAvh171R |
| | | | and PrimerD |
| UNM1 | pUN and M1 | Ham34::Avr1b(RxLR1) inserted | Selection and Gus expression |
| | | together with rpL41::NptII for P. sojae | cassettes in pCambia1305.2 |
| | | transformation | replaced by inserts from pUN and |
| | | | M1, respectively |
| UNM2 | pUN and M2 | Ham34::Avr1b(RxLR2) inserted | Selection and Gus expression |
| | | together with rpL41::NptII for P. sojae | cassettes in pCambia1305.2 |
| | | transformation | replaced by inserts from pUN and |
| | | | M2, respectively |
| UNM1 + 2 | pUN and M1 + 2 | Ham34::Avr1b(RxLR1 + 2) inserted | Selection and Gus expression |
| | | together with rpL41::NptII for P. sojae | cassettes in pCambia1305.2 |
| | | transformation | replaced by inserts from pUN and |
| | | | M1 + 2, respectively |
| UNM3 | pUN and M3 | Ham34::Avr1b(dEER) inserted | Selection and Gus expression |
| | | together with rpL41::NptII for P. sojae | cassettes in pCambia1305.2 |
| | | transformation | replaced by inserts from pUN and |
| | | | M3, respectively |
| pCambiaAvr1b | pCambia1305.2 | CaMV 35S promoter fused to | HMT gene in pCambia1305.2 |
| | | Avr1b in GUS-containing vector | replaced by Avr1b-1 using |
| | | | primers Avr1bfull F and Avr1bR |
| pCambiaM2 | pCambia1305.2 | CaMV 35S promoter fused to | HMT gene in pCambia1305.2 |
| | | Avr1b(RXLR2) in GUS-containing | replaced by Avr1b(RXLR2) using |
| | | vector | primers Avr1bfull F and Avr1bR |
| pCambia- | pCambia1305.2 | CaMV 35S promoter fused to | HMT gene in pCambia1305.2 |
| mAvr1b | | leaderless Avr1b in GUS- | replaced by mAvr1b-1 using |
| | | containing vector | primers Avr1bF and Avr1bR |
| pCambia-mM2 | pCambia1305.2 | CaMV 35S promoter fused to | HMT gene in pCambia1305.2 |
| | | leaderless Avr1b(RXLR2) in GUS- | replaced by mAvr1b(RXLR2 |
| | | containing vector | using primers Avr1bF and |
| | | | Avr1bR |
| pCa-GUS(—) | pCambia1305.2 | Empty vector as the control | GUS expression cassette was |
| | | | removed by Sph I restriction and |
| | | | re-ligation for co-transformation |
| | | | experiments |
| pCaAvr1b | PCambiaAvr1b | CaMV 35S promoter fused to | |
| | | Avr1b in GUS-free vector | |
| pCaM2 | pCambiaM2 | CaMV 35S promoter fused to | |
| | | Avr1b(RXLR2) in GUS-free | |
| | | vector | |
| pCa-mAvr1b | pCambia-mAvr1b | CaMV 35S promoter fused to | |
| | | leaderless Avr1b in GUS-free | |
| | | vector | |
| pCa-mM2 | pCambia-mM2 | CaMV 35S promoter fused to | |
| | | leaderless Avr1b(RXLR2) in GUS- | |
| | | free vector | |

P. sojae transformants were selected that grew well on V8 medium with 50 ug/ml G418, and were cultured in V8 liquid medium for 3 days. The mycelia were harvested frozen in liquid nitrogen and ground to a powder for DNA or RNA extraction. Genomic DNA was isolated from mycelium as described by Judelson et al. (1991) with extraction buffer. DNA samples were quantified using a Nanodrop ND-1000 spectrophotometer. The presence of Avr1b-1 transgenes was verified by PCR amplification from 100 ng genomic DNA using a program of 94° C. for 2 min, 30 cycles of 94° C. for 30 s, 56° C. for 30 s, 72° C. for 30 s, and 72° C. for 5 min. The primers used were HamF (5′-TTCTCCTTTTCACTCTCACG-3′; SEQ ID NO:20) and HamR (5′-AGACACAAAATCTGCAACTTC-3′; SEQ ID NO:21). PCR products were restricted with Pst I and/or sequenced. The Pst I restriction profile of unmodified Avr1b-1 is a 577 bp fragment, of Avr1b (RxLR1) is 215 bp and 362 bp, of Avr1b (RxLR2) is 248 bp and 329 bp, of Avr1b (RxLR1+2) is 33 bp, 215 bp, and 329 bp and of Avr1b (dEER) is 266 bp and 311 bp.

RNA was extracted from each sample using RNeasy Plant Mini Kit (QIAGEN, Cat. No. 74904) with β-Mercaptoethanol added buffer RLT according to the manufacturer's recommendations. Genomic DNA was removed using RNase-Free DNase (QIAGEN, Cat. No. 79254) according to the manufacturer's recommendations. RNA was quantified using a Nanodrop ND-1000 spectrophotometer. Avr1b-1 transgene transcription was verified by RT-PCR with primers, Avr1bReF (5′-ACCTTCAGCGTGACTGACCT-3′; SEQ ID NO:22) and Avr1bReR (5′-GCGATTGCCAACCAGTTCT-3′; SEQ ID NO:23) which are internal to the Avr1b-1 gene and downstream of the mutation sites. As a reference, P. sojae actin mRNA was measured using primers ActinReF (5′-GGCGAGCGTATGACCAAG-3′; SEQ ID NO:24) and ActinReR (5′-GAACCACCGATCCAGACC-3′; SEQ ID NO:25). The primers were designed using Beacon Design 4.0 (Primer Biosoft International, Palo Alto, Calif.) and synthesized by Integrated DNA Technologies, Coralville, Iowa. In each case, 100 ng total RNA was reverse transcribed using SuperScript™ III First-Strand Synthesis System for RT-PCR (Invitrogen, Cat. No: 18080-051) with an oligo(dT)₂₀ primer using the provided protocols and then amplified by Taq polymerase using the following program: 94° C. for 2 min, 30 cycles of 94° C. for 30 s, 56° C. for 30 s, 72° C. for 30 s, and 72° C. for 5 min. As a control for the presence of contaminating genomic DNA template, the SuperScript™ III RT was omitted. Transformants in which Avr1b-1 transgene expression could be detected by RT-PCR were selected for further characterization by quantitative RT-PCR assay and by avirulence testing.

The expression of the Avr1b-1 transgene in transformed P. sojae lines was further validated by quantitative RT-PCR. Primers were dissolved to a concentration of 40 uM using RT-grade PCR water (Ambion Inc., Austin, Tex.). RNA samples were assessed for quality and quantity on the Agilent Bioanalyzer 2100 (Agilent Technologies, Santa Clara, Calif.) and Nanodrop Spectrophotometer (Nanodrop Technologies, Wilmington, Del.), respectively. Total RNA (lug) was then transcribed to cDNA using Invitrogen first strand cDNA synthesis reagents (Invitrogen Corp., Carlsbad, Calif.) in a total volume of 20 ul. Each cDNA was diluted 1:10 with RT grade PCR water. Standard curves were produced with serial 10 fold dilutions of purified, amplified cDNA products starting with 10 pg/ul. Quantitative PCR reactions consisted of 300 nM sense and anti-sense primers, 1 ul diluted cDNA, and RT-grade PCR water to obtain a volume of 12.5 ul. SYBR-Green I PCR Mastermix (Applied Biosystems, Foster City, Calif.) was added to obtain a final reaction volume of 25 ul. Each reaction was run in triplicate for both the standard and unknown samples. Reactions were run under the following conditions on the Bio-Rad I-cycler (BioRad. Hercules, Calif.): 95° C. denaturation for 3 min, 40 cycles of 95° C. for 10 s, 56° C. for 45 sec to calculate cycle threshold values, followed by 95° C. for 1 min, 55° C. for 1 min, and 80 times of 55° C. for 10 s, increasing temperature by 0.5° C. each cycle to obtain melt curves. The relative amount of all mRNAs was calculated using the comparative threshold cycle (CT) method and P. sojae actin mRNAs were used as the invariant controls.

Avr1b phenotypic expression was assayed using soybean cultivars HARO(1-7) (rps), Haro13 (Harosoy background, Rps1b), Williams (rps) and L77-1863 (Williams background, Rps1b). Seedlings were grown in the green house or in a growth chamber (Percival AR-36L) with a program of 24° C. at daytime and 22° C. at night with a 14-hrs day length under fluorescent light (250 μmol photons s⁻¹ m⁻²).

The virulence of each transformant was evaluated using hypocotyl inoculation (Forster et al. 1994). 1-2 days after the first primary leaf appeared, the hypocotyl of the soybean was wounded with a short incision and the incision was inoculated with a small piece of V8 agar cut from the edge of a 3 day old colony. Thereafter, the plants were incubated in a growth chamber under the conditions described above. The numbers of dead and surviving plants were counted 4 days after inoculation, and summed over 2-5 replicates. The differences between the numbers of surviving plants from rps and Rps1b cultivars were compared using Fisher's exact test and p value was calculated as p=[(a+b)!(c+d)!(a+c)!(b+d)!]/[(a+b+c+d)!a!b!c!d!] (a, number of surviving seedlings of Rps1b cultivars; b, number of died seedlings of Rps1b cultivars; c, number of surviving seedlings of rps cultivars; and d, number of died seedlings of rps cultivars). Transformants producing no significant difference between rps and Rps1b cultivars were judged virulent, and those with a significant difference were judged avirulent.

An embodiment of the device of the present invention was used for particle bombardment assays. Soybean plants were grown as described in the previous section, except that in the growth chamber, the day was 12 hours at 28° C. and the night 12 hours at 25° C. The first (monofoliate) true leaves were selected for bombardment 9-14 days after planting. Plasmid DNA was isolated using Qiagen brand Maxi Preparation Kits and concentrated to 5-6 ug/ul. M-10 tungsten particles were washed twice with 95% ethanol, twice with sterile deionized water and then resuspended in 50% sterile glycerol to a concentration of 90 mg/ml. For bombardment, 9 mg of tungsten particles were combined with 50 ug of GUS plasmid DNA (pCambia1305.2) and 50 ug of test DNA (pCaAvr1b, pCaM2, pCa-mAvr1b, pCa-mM2 or empty vector pCa-GUS(−) as the control) in a total of 100 ul of 25% glycerol on ice in a 0.5 ml centrifuge tube. 65 ul of 2.5M CaCl₂ was added followed by 25 ul of freshly prepared 0.1 M spermidine. The preparation was vortexed for 2 minutes then placed on ice for 20 minutes. The particle preparation was then concentrated to 30 ul by brief centrifugation.

Bombardment was carried out using the Bio-Rad He/1000 Particle Delivery System to which was attached a double-barreled extension (FIGS. 1 and 2) that enables leaves to be bombarded with two DNA preparations simultaneously. One microliter of each DNA-particle preparation was loaded onto the macrocarrier so that the mixtures were directly over their respective barrels (FIG. 1C). The distance from the stopping screen to the target shelf was 12 cm. The distance between the rupture disk and macrocarrier was set to ⅜″ (9.5 mm) and the highest position was used for the macrocarrier in the macrocarrier assembly. 650 psi rupture disks were used. The chamber vacuum was 26 psi. The pressure build time was set to 12-14 seconds. The two barrels of the extension were cleaned with 70% ethanol, and then dried with compressed air between shots.

The target leaves were bombarded twice, first the petiole-distal half of the leaf then the petiole-proximal half, resulting in a total of four bombardment sites. A cover was used to prevent overlapping bombardments. A total of 16 pairs of bombardments were performed on 8 leaves expressing the Rps1b gene (8 bombardments, two bombardments on each leaf) and on control leaves lacking Rps1b. The leaves expressing Rps1b and control leaves were alternated every 4 bombardments throughout each experiment.

After bombardment, the leaves were incubated for 5 days in darkness at 28° C. The leaves were then stained for 16 hours at 28° C. using 0.8 mg/ml X-gluc (5-bromo-4-chloro-3-indolyl-beta-D-glucuronic acid, cyclohexylammonium salt), 80 mM Na phosphate pH 7.0, 0.4 mM K₃Fe(CN)₆, 0.4 mM K₄Fe(CN)₆, 8 mM Na₂EDTA, 0.8 mg/ml, 20% methanol, 0.06% (v/v) Triton X-100, and then de-stained in 100% methanol. Blue spots were counted using a dissecting microscope at 5×-20× magnification.

The avirulence activity of the Avr1b-1 constructs was measured as the reduction in the number of blue spots comparing the Avr1b-1+GUS bombardment with the GUS+control bombardment. For each paired shot, the logarithm of the ratio of the spot numbers of Avr1b-1 to that of the control was calculated, then the log-ratios obtained from the Rps1b and non-Rps1b leaves were compared using the Wilcoxon rank sum test.

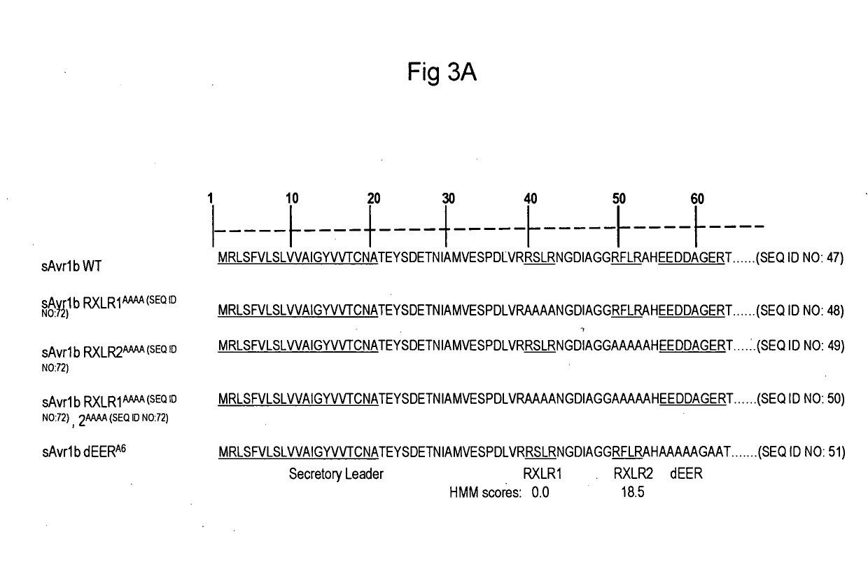

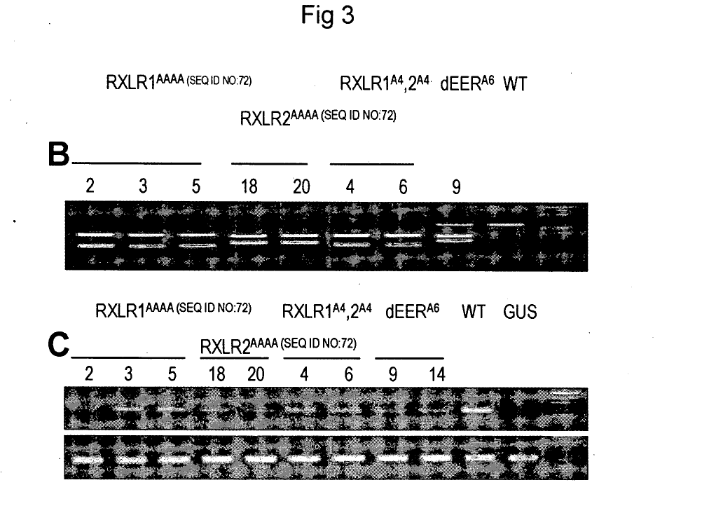

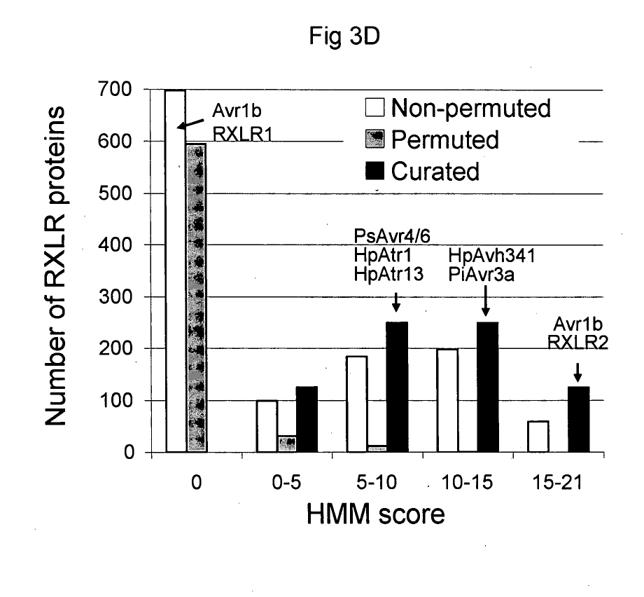

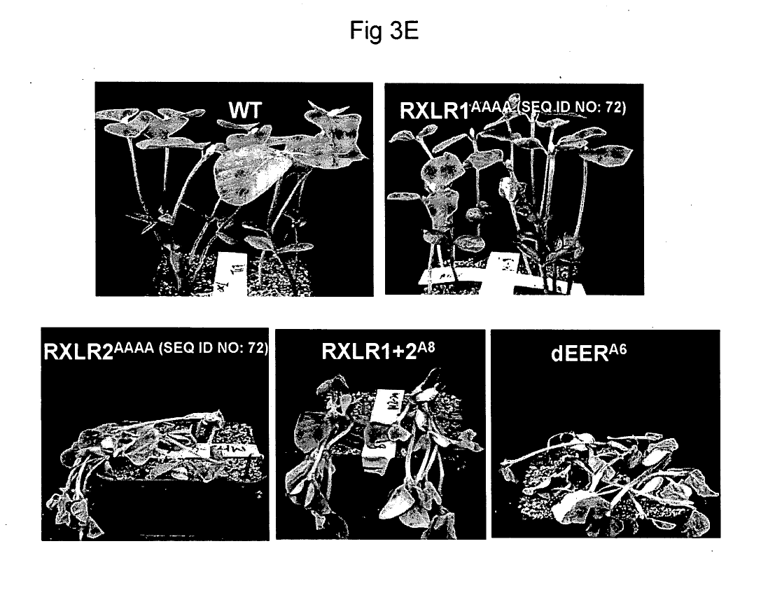

Oomycetes are fungal-like organisms that are evolutionarily related to marine algae. Many oomycete species are destructive plant pathogens, for example the potato late blight pathogen, Phytophthora infestans, that caused the Irish Potato Famine, the Sudden Oak Death pathogen, P. ramorum, and the soybean pathogen P. sojae. Many bacterial pathogens of plants and animals deliver effector proteins into host cells using the type III secretion pathway. Plant disease resistance genes that provide protection against oomycetes encode intracellular receptors, implying that pathogen proteins (called avirulence proteins) that are recognized by these receptors also can enter the cytoplasm of their plant hosts, but the possible mechanism of entry is unknown. Four oomycete avirulence genes have been cloned, Avr1b-1 from P. sojae (Shan et al., 2004), Avr3a from P. infestans (Allen et al., 2004), and ATR1 (Rehmany et al., 2005) from the Arabidopsis downy mildew pathogen Hyaloperonospora parasitica. In the cases of Avr3a, ATR1 and ATR13, expression of the encoded proteins inside the host cytoplasm resulted in recognition of the avirulence proteins by the plant receptors, confirming that the cytoplasm is the site of interaction between the resistance gene receptors and the avirulence proteins. Because the avirulence proteins appear to enter the host cells, they have been inferred to be effector proteins. Comparison of the sequences of the four cloned avirulence genes with each other and with large diverse families of similar genes in the P. sojae and P. ramorum genome sequences (Avh genes) identified two conserved motifs, termed RXLR1, RXLR2 and dEER near the N terminus of these secreted proteins (Tyler et al., 2006; Birch et al., 2006, Rehmany et al., 2005) (FIG. 3A).

More specifically, FIG. 3 shows that the RXLR and dEER motifs are required for Avr1b function in P. sojae transformants. Panel A shows the sequences of mutations in the RXLR1, RXLR2 and dEER motifs. Panel B shows Pst I restriction analysis of PCR products amplified from Avr1b-1 transformants using primers specific for the HAM34 promoter and terminator regions. Pst I restriction profiles of Avr1b(RXLR1^{AAAA}), Avr1b(RXLR2^{AAAA}), Avr1b(RXLR1^{AAAA}, 2^{AAAA}), Avr1b(dEER^{A6}) and wild type (WT) Avr1b are distinguished from each other because the mutations introduce a Pst I site. Avr1b(dEER^{A6})-9 was confirmed by sequencing the PCR product. Panel C shows the detection of Avr1b mRNA in P. sojae stable transformants by RT-PCR. The upper panel shows amplification with primers internal to the Avr1b C-terminus. The lower panel shows amplification with P. sojae actin primers. P. sojae stable transformants were the same as for Panel B except that an amplification reaction is also shown from RNA from a P. sojae transformant containing a β-glucuronidase gene (GUS). No amplification was observed when reverse transcriptase was omitted from the reactions (not shown). Panel D shows the distributions of HMM scores of RXLR flanking regions for all RXLR-containing secreted proteins from P. sojae and P. ramorum (non-permuted), for all secreted proteins retaining an RXLR string after sequence permutation (permuted), and for all high quality RXLR-effector candidates identified by Jiang et al (2008) (curated). The locations on the distribution of the HMM scores of the RXLR strings of known avirulence proteins, and HpAvh341 are shown by the arrows. Panel E depicts the phenotype of L77-1863 (Rps1b) seedlings inoculated on the hypocotyls with transformants carrying the indicated wild type or mutant Avr1b-1 genes and photographed four days later.

The RXLR motif closely resembles a motif (Pexel or VTF; RXLX^{E}/_{Q}) that enables Plasmodium effector proteins to cross the parasitiphorous vacuolar membrane into the cytoplasm of erythrocytes. Furthermore, the RXLR motif of Avr3a was shown to function in targeting proteins from malaria parasites into the erythrocyte cytoplasm (Bhattacharjee et al., 2006). The structural and functional similarity between the RXLR and Pexel/VTF motifs had encouraged the hypothesis that the RXLR motif was responsible for transit of the oomycete effector proteins into the cytoplasm of host cells. The experiments described here, including the use of an embodiment of the device of the present invention, experimentally verified this hypothesis and also demonstrated that RXLR-mediated transit does not require presence of the pathogen.

To test the function of the RXLR and dEER motifs of Avr1b-1, we created transgenic P. sojae strains using protoplast fusion (Dou et al., 2008a) which expressed wild-type and mutant Avr1b-1 genes. Avr1b-1 carries two RXLR motifs, so we created mutations in either or both of the RXLR motifs, in addition to a mutation in the dEER motif (FIG. 3A). Two independent transformants expressing wild type Avr1b-1 acquired avirulence against Rps1b, as expected; that is, they lost the ability to infect soybean plants carrying Rps1b, but were unaffected in their ability to infect plants lacking Rps1b (FIG. 3E). This was confirmed using two different isolines of soybean, which differed only in the presence of Rps1b, namely Williams (nor Rps) and L77-1863 (Rps1b; Williams background) and HARO(1-7)1 (No Rps; Harosoy background) and HARO13 (Rps1b; Harosoy background). In five independent transformants expressing the RXLR2 mutant however, there was no gain of avirulence on Rps1b cultivars, despite the presence of abundant mRNA from the transgene (FIG. 3C). Thus the RXLR2 motif is required for Avr1b activity when the protein is delivered by the pathogen, which does not pierce the plant plasma cell membrane. Because the second RXLR motif, RXLR1, was intact in the RXLR2 mutant, it appeared to be non-functional. Consistent with this inference, RXLR1 mutations did not abolish avirulence in three independent transformants (FIG. 3E). As expected, avirulence was lost in the RXLR1; RXLR2 double mutants (FIG. 3E). A mutation in the dEER motif also abolished avirulence (in two independent transformants) indicating that this motif is also required for the function of the protein (FIG. 3E). The difference in activity between RXLR1 and RXLR2 suggests that surrounding sequences are important to the activity of an RXLR motif. We previously created a hidden markov model (HMM) using the sequences surrounding the RXLR motifs of all of the P. sojae Avh genes (Tyler et al., 2006). The sequences surrounding RXLR2 scored highly (18.5) with the HMM whereas those surrounding RXLR1 scored more poorly (0.0), demonstrating that the sequences surrounding the RXLR motifs have a restricted composition that is important to the targeting function.

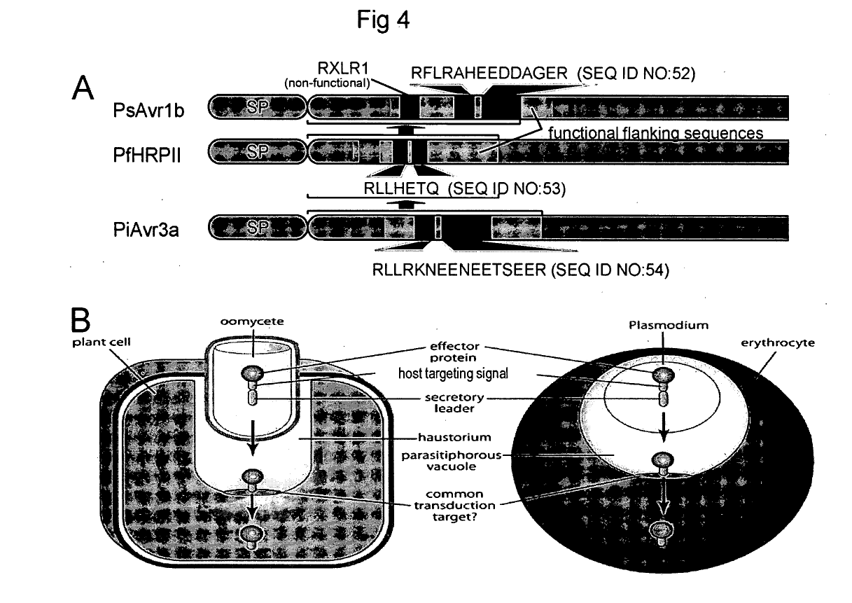

FIG. 4 depicts a common host targeting mechanism in oomycetes and Plasmodium. Panel A shows the features and functional exchange of host targeting signals in Ph. sojae Avr1b, Pl. falciparum HRPII and P. infestans Avr3a. Common RxL(R) and dEER-like motifs are shown, and were defined experimentally (Dou et al, 2008b; Marti et al., 2004; Hiller et al., 2004; Whisson et al., 2007). Flanking regions inferred also to be required are shown. In PsAvr1b, the flanking regions are defined as the 19 residues upstream and 16 residues downstream of RXLR2 shown to be sufficient for translocation in GFP fusion experiments. In PfHRPII, the 8 residues upstream and 13 residues downstream were defined experimentally (Bhattacharjee et al., 2006). In PiAvr3a, the flanking regions are defined by the region tested in Plasmodium (Bhattacharjee et al, 2006). The region of PiAvr3a tested in Plasmodium and the region of PfHRPII tested in P. sojae are shown by the brackets.

Panel B of FIG. 4 shows that the anatomical contexts of oomycete and Plasmodium effector entry are similar. The haustorium is a specialized invagination of the plant cell formed by oomycete (and fungal) pathogens. The plant cell wall is pierced during formation of the haustorium, while the oomycete cell wall is retained but differentiates into the haustorial wall. Both the haustorial membrane and the parasitiphorous vacuolar membrane are derived from the host plasma cell membrane during pathogen invasion.

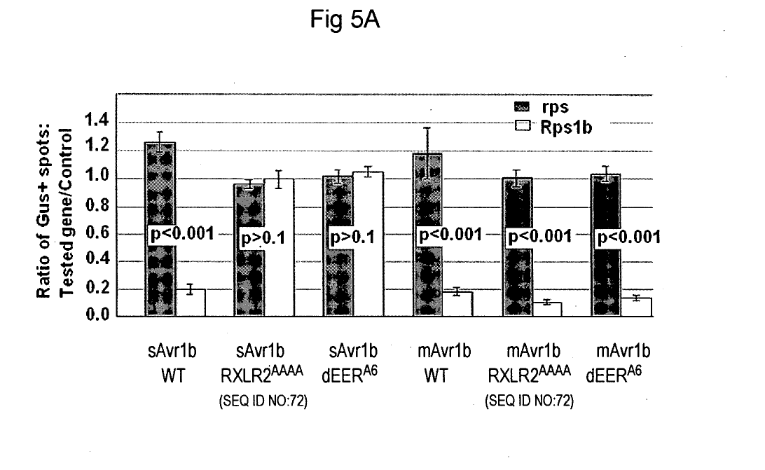

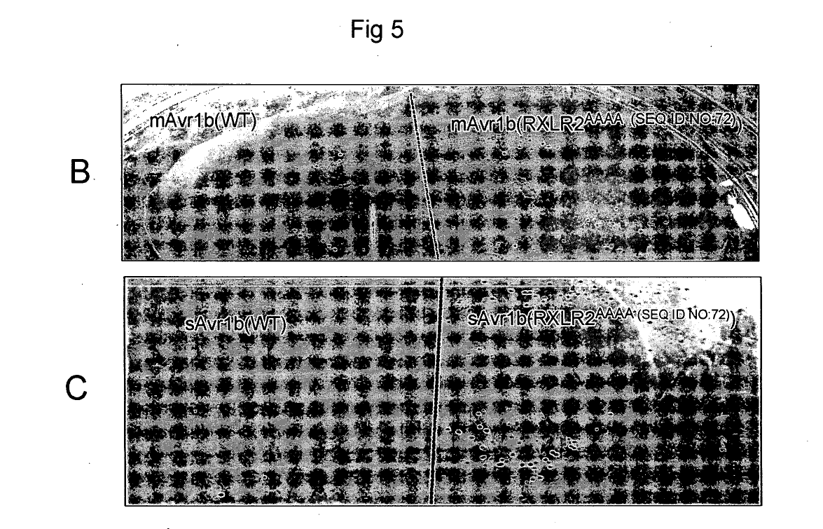

To support the conclusions from the transgenic P. sojae experiments, and to test whether RXLR function requires the presence of the pathogen, we introduced DNA encoding Avr1b proteins into soybean cells together with DNA encoding 13-glucuronidase (GUS) using particle bombardment with the device of the invention (FIG. 5). This assay measures the functional interaction of the Avr1b protein with the intracellular product of the soybean Rps1b gene; when the two proteins interact, programmed cell death is triggered in the transformed cells ablating the development of tissue patches expressing GUS. Soybean leaves were bombarded using a double-barreled device of the current invention that delivered Avr1b-1 DNA-bearing particles to one side of the leaf and control (empty vector) DNA to the other; both sides received GUS DNA. Panel A shows the ratio of blue spots in the presence of Avr1b-1 compared to the control. sAvr1b indicates a gene encoding secretory Avr1b and mAvr1b indicates one encoding mature Avr1b (lacking the secretory leader). WT indicates wild-type RXLR motif, RXLR2^{AAAA} indicates the four alanine replacement of the RXLR2 motif, dEERA6 indicates the six alanine replacement of the dEER motif. Averages and standard errors are from 16 pairs of shots. p values comparing results from cultivars with Rps1b (L77-1863) or without (rps; Williams) were calculated using the Wilcoxon rank sum test. Panels B and C depict a direct comparison of bombardment with mature Avr1b and secretory Avr1b, respectively. In both Panels B and C, DNA encoding WT (left) and RXLR2^{AAAA} (right) versions of mAvrb or sAvr1b, respectively, were bombarded onto the same leaf of L77-1863 (Rps1b). The dashed lines indicate the positions of a divider that prevents particles from the two shots from overlapping. In both photographs, the brightness and contrast were adjusted uniformly to improve the visibility of the spots.

FIG. 5 shows that delivery of Avr1b-1 DNA into soybean cells significantly reduced the number of blue GUS-positive patches when the Rps1b gene was present, but not when Rps1b was absent. When the secretory leader of Avr1b was deleted, the interaction of Avr1b protein with the Rps1b gene product still occurred, consistent with a cytoplasmic location for the Avr1b-Rps1b interaction. When RXLR2 was replaced by four alanine residues, the interaction of the cytoplasmic, leader-less Avr1b with Rps1b was unaffected, indicating that the RXLR motif was not required for the interaction. However, mutation of RXLR2 prevented the interaction when the secretory leader was present. From this result, we infer firstly that the secretory leader is functional in soybean and targets Avr1b protein to the outside of the cell, and secondly that the RXLR2 motif is required for Avr1b protein to re-enter the cell. As in the P. sojae transgenics, the presence of the RXLR1 motif was unable to complement the RXLR2 mutation.

Taken together, our findings that the RXLR2 motif of Avr1b is required for avirulence function when the protein is delivered to the outside of the host plant cell, but not when it is delivered to the inside, strongly support the hypothesis that this motif is responsible for transit of the protein across the host plasma cell membrane. The dEER motif and sequences surrounding the RXLR motif are also required.

The Avr1b protein requires not only the RXLR motif itself, but also surrounding sequences including the dEER motif. This requirement is shared by the Plasmodium Pexel/VTF motif (Bhattacharjee et al., 2006). In both cases, these surrounding sequences are not enriched in positive and hydrophobic residues, but rather are enriched in acidic and hydrophilic residues. The Plasmodium Pexel/VTF motif, including the surrounding sequences, can be replaced by the oomycete RXLR domain. Therefore oomycete and Plasmodium effector transduction might share a novel mechanism, different from that of animal PTDs, that targets host cell surface machinery that is common to plants and vertebrate animals.

### Example 2

### Insertion of Plasmid DNA into Plant Cells

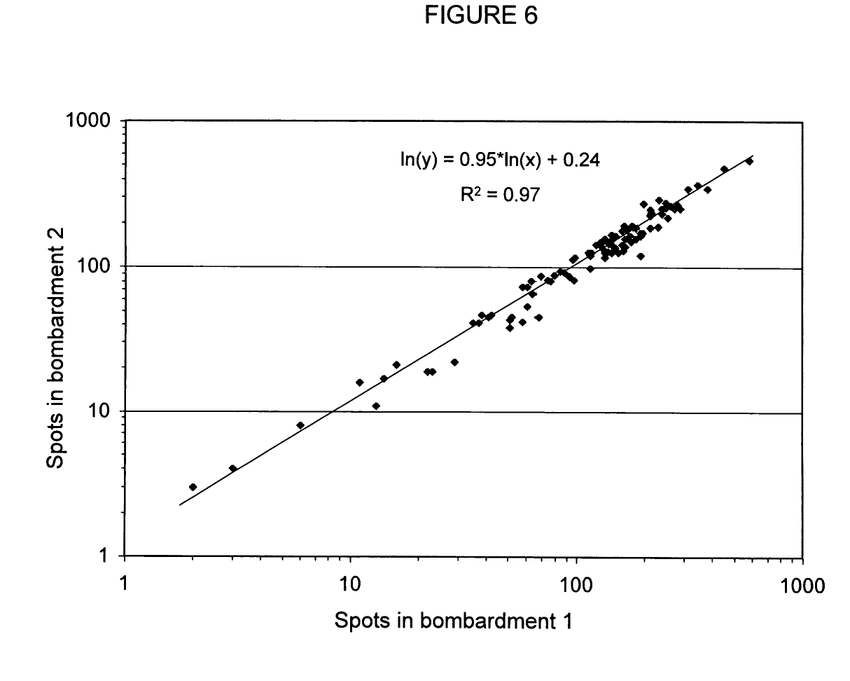

As another demonstration of the effectiveness of the attachment or device of the invention, plasmid DNA encoding the GUS gene was coated onto tungsten particles and then shot into 13 soybean leaves using the double-barreled device attached to the above-referenced Bio-Rad device. The results are shown in Table 3 and FIG. 6. A great variability of DNA introduction between leaves is evident, with a range from 3 to 176 transformed cells observed. However, the reproducibility between the barrels is excellent, yielding a correlation coefficient (r²) of 0.94.

TABLE 3

| Barrel 1 | Barrel 2 |
|---|---|
| 7 | 3 |
| 10 | 7 |
| 12 | 4 |
| 14 | 24 |
| 30 | 44 |
| 33 | 35 |
| 52 | 45 |
| 61 | 72 |
| 62 | 40 |
| 65 | 50 |
| 115 | 104 |
| 122 | 96 |
| 174 | 176 |

More specifically, plasmid DNA encoding the GUS gene was coated onto tungsten particles and then shot into 13 soybean leaves using an embodiment of the device of the invention. Three days after bombardment, the leaves were stained for GUS enzyme activity and the numbers of blue spots counted. The rows of Table 3 show the results for the 13 leaves. The two columns compare the results for the two barrels on each leaf. The graph depicted in FIG. 6 plots the results for Barrel 1 against those for Barrel 2. As can be seen from the graph, there is a good correlation between the amount of DNA introduced and expressed in leaf cells from each barrel.

As part of the present invention, the preparation of tungsten for bombardment has been modified from a common known preparation technique. However the modifications made are not always necessary for accurate results, although they are optimal for some instances. Based on experimental results, it is not preferred to use dry preparations, such as those currently used in the Bio-Rad system. Other optimization results for certain embodiments include: use of a final spermidine concentration of 15 mM, use of a final calcium chloride concentration of 1 M, use of an amount of nucleic acid of 50 ug per preparation (although the amount of nucleic acid, e.g., DNA, can vary). In some embodiments, it has been found that 100 ul of 90 mg/ml Tungsten in 50% glycerol can be used, vortexed, and 50 ul removed for use. Although not required, the DNA for transformation may be precipitated, such as for 20 minutes, then centrifuged. In some experiments, a final volume of 30 ul was used advantageously.

One experiment focused on optimization of parameters for a nucleic acid and plant cell combination (Tables 4, 5, and 6). More specifically, optimization of the following parameters was determined: rupture disk pressure, size of tungsten particle, volume of bombardment preparation. It was determined that an advantageous setup included use of 26 PSI vacuum and a rubber holder, a rupture pressure of 650 psi, and a particle size of M-10 tungsten from Bio-Rad, and a volume of bombardment prep of 1 ul. Table 6 depicts data produced from a Custom Build metal holder which allows for barrels to be held in the same exact position for each bombardment.

TABLE 4

| PSI | Tungsten | Amount (ul) | GUS Spots |
|---|---|---|---|
| 650 | M-10 | 0.5 | 0 |
| 650 | M-10 | 0.5 | 0 |
| 650 | M-10 | 1 | 31 |
| 650 | M-10 | 2 | 13 |
| 450 | M-10 | 0.5 | 0 |
| 450 | M-10 | 1 | 0 |
| 450 | M-10 | 1 | 0 |
| 450 | M-10 | 1 | 0 |
| 450 | M-10 | 2 | 0 |
| 450 | M-10 | 2 | 0 |
| 650 | M-5 | 0.5 | 0 |
| 650 | M-5 | 0.5 | 0 |
| 650 | M-5 | 1 | 0 |
| 650 | M-5 | 1 | 0 |
| 650 | M-5 | 2 | 0 |
| 450 | M-5 | 0.5 | 0 |
| 450 | M-5 | 1 | 0 |
| 450 | M-5 | 1 | 0 |
| 450 | M-5 | 1 | 0 |

TABLE 5

| Shot | | Upper Barrel | Lower Barrel GUS | Percent |
|---|---|---|---|---|
| Number | PSI | GUS Spots | Spots | difference |
| 1 | 650 | 0 | 0 | 0 |
| 2 | 650 | 23 | 29 | 23 |
| 3 | 650 | 211 | 107 | 65 |
| 4 | 650 | 29 | 37 | 24 |
| 5 | 650 | 70 | 62 | 12 |
| 6 | 650 | 80 | 63 | 24 |
| 7 | 650 | 160 | 53 | 100 |
| 8 | 650 | 123 | 139 | 12 |

TABLE 6

| | | | | | | GUS | |
|---|---|---|---|---|---|---|---|
| | | | GUS | | | Spots | |
| | | | Spots | | | Closer | |
| | Region | Plasmid | Closer to | | Plasmid | to Tip | |
| Soybean | Shot | for Stem | Stem | Percent | for Tip | of | Percent |
| Cultivar | First | Area | Area | Difference | Area | Leave | Difference |
| Haro 13 | Tip | GUS | 26 | 4 | GUS | 65 | 5 |
| | | GUS | 25 | | GUS | 68 | |
| Haro 13 | Tip | GUS | 35 | 11 | GUS | 283 | 3 |
| | | GUS | 39 | | GUS | 275 | |
| Haro 1-7 | Tip | GUS | 162 | 9 | GUS | 79 | 21 |
| | | GUS | 148 | | GUS | 64 | |
| Haro 1-7 | Tip | GUS | 38 | | GUS | 153 | |
| | | GUS | 78 | | GUS | 146 | 5 |

In one set of experiments, the bombardment preparations were not correctly aligned with the barrels and it was determined that there is a significant difference in effectiveness when the barrels are not aligned with the preparation.

### Example 3

### Use of the Device of the Invention (DoubleShot Gene Gun) to Characterize the Properties of Other Proteins

Plasmids and strains were constructed as described below. Soybean transient expression plasmids pUCAvr1b (leaderless Avr1b-1 driven by CaMV 35 S promoter) and pUCGUS (GUS gene driven by CaMV 35 S promoter) were constructed as follows. For pUCGUS, a cassette containing the CaMV 35S promoter, CAMBIA GUSPlus gene and nos terminator was amplified from pCambia1305.2 (AF354046.1) using primers GusF_EcoRI and GUSR_HindIII (Table 7), cleaved with EcoRI and HindIII and ligated into pUC19. For pUCAvr1b, the hygromycin resistance gene of pCambia1305.2 was replaced by Avr1b-1 (AF449620.1) lacking its secretory leader (replaced by ATG codon) using primers Avr1bF and Avr1bR (Table 7), creating pCambia-mAvr1b. Then the Avr1b expression cassette, including the double 35S promoter was amplified from pCambia-mAvr1b using primers Avr1b_EcoRI, Avr1b_HindIII and Avr1b_genegun_KpnI (Table 9) and subcloned into pUC19(M77789.2). These primers also created XmaI and KpnI sites for later manipulation of the Avr1b-1 gene. Plasmids pUCBax was constructed by insertion into Xma I- and Kpn I-digested pUCAvr1b with the amplicon obtained with oligonucleotides: BaxF and BaxR (Table 7). In Table 7, uppercase letters indicate bases that match the initial template. Lower case letters indicate mutations or 5′ extensions that do not match the initial template. Restriction sites introduced into the amplicon are underlined and start or stop codons are both in bold.

TABLE 7

| | | |
|---|---|---|
| Name | Sequences (from 5′ to 3′) | Usage |

| | | |
|---|---|---|
| Avr1bF | atcgcactcgagctttcgcagatcccggggggcaatgagatatgACTGAGT | For insertion of |
| | ACTCCGACGAA | Avr1b-1 genes into |
| | (SEQ ID NO:26) | plant transient |
| Avr1bR | gaaactcgagcttgtcgatcgacagatccggtcggcaggtaccTCAGCTCT | expression assay |
| | GATACCGGTG | vector, |
| | (SEQ ID NO:27) | pCambia 1305.2 |
| BaxF | agatcccggggggcaatgagatATGGACGGGTCCGGGGAG | Insertion of BAX |
| | (SEQ ID NO:28) | gene into soybean |
| BaxR | ggcaggtaccTCAGCCCATCTTCTTCCAG | transient expression |
| | (SEQ ID NO:29) | assay vector |
| GusF_EcoRI | TCCCGaaTTCAGTTTAGCTTCATG | Transfer of GUSPlus |
| | (SEQ ID NO:30) | expression cassette |
| GusR_HindIII | TAGTaagcTTCCCGATCTAGTAACAT | from pCambia 1305.2 |
| | (SEQ ID NO:31) | to pUC19 |
| Avr1b_EcoRI | GGAGgaaTTcGCTGGCTGGTGGCAGGAT | Transfer of Avr1b |
| | (SEQ ID NO:32) | expression cassette |
| Avr1b_HindIII | GTATTGGCTAGAGaAGCTTGCCA | from pCambia 1305.2 |
| | (SEQ ID NO:33) | into pUC19 vector |
| Avr1b_gene | AGAAACTCGAGCTTGTCGATCGACAGATCCGGTCG | with addition of Xma I |
| gun_KpnI | GCAggTACcTCAGCTCTGATAC | and Kpn I sites |
| | (SEQ ID NO:34) | flanking Avr1b-1 gene |

Particle bombardment assays for avirulence and virulence phenotypes were performed as described using the device of the current invention. Soybean plants were grown as described in the previous section, except that in the growth chamber, the day was 12 hr at 28° C. and the night was 12 hr at 25° C. The first (monofoliate) true leaves were selected for bombardment 9-14 days after planting.

Plasmid DNA was isolated using Qiagen brand Maxi Preparation Kits and concentrated to 5-6 ug/ul in sterile deionized water. M-10 tungsten particles (Bio-Rad, Hercules, Calif.) were washed twice with 95% ethanol and twice with sterile deionized water, then resuspended in 50% sterile glycerol to a concentration of 90 mg/ml. For bombardment, 9 mg of tungsten particles were combined with 50 ug of GUS plasmid DNA (pCambia 1305.2) and 50 ug of either test DNA (e.g., pCaAvr1b) or empty vector (pCa-GUS(−) or pUC19) as the control in a total of 100 ul of 25% glycerol on ice in a 0.5 ml centrifuge tube. 65 ul of 2.5M CaCl₂ was added followed by 25 ul of freshly prepared 0.1 M spermidine. The preparation was vortexed for 2 minutes, then placed on ice for 20 min. The particle preparation was then concentrated to 30 ul by brief centrifugation. Bombardment was carried out using the Bio-Rad He/1000 Particle Delivery System with the DoubleShot device attached (Dou et al., 2008a). 1 ul of each DNA-particle preparation was loaded onto the macrocarrier so that the mixtures were directly over the respective barrels. The distance from the stopping screen to the target shelf was 12 cm. The distance between the rupture disk and macrocarrier was set to ⅜ inch (9.5 mm), and the highest position was used for the macrocarrier in the macrocarrier assembly. 650 psi rupture disks were used. The chamber vacuum was 26 psi. The pressure build time was set to 12-14 seconds. The two barrels of the extension were cleaned with 70% ethanol, then dried with compressed air between shots. The target leaves were bombarded twice, first the petiole-proximal half of the leaf then the petiole-distal half, resulting in a total of four bombardment sites. A cover was used to prevent overlapping bombardments. A total of 16 pairs of bombardments were performed on 8 leaves expressing the Rps1b gene (8 bombardments, 2 bombardments on each leaf) and on control leaves lacking Rps1b. The leaves expressing Rps1b and the control leaves were alternated every 4 bombardments throughout each experiment. After bombardment, the leaves were incubated for 5 days in darkness at 28° C. The leaves were then stained for 16 hr at 28° C. using 0.8 mg/mL X-gluc (5-bromo-4-chloro-3-indolyl-beta-D-glucuronic acid, cyclohexylammonium salt), 80 mM Na phosphate pH 7.0, 0.4 mMK₃Fe(CN)₆, 0.4 mM K₄Fe(CN)₆, 8 mM Na₂EDTA, 0.8 mg/ml, 20% methanol, 0.06% (v/v) Triton X-100, and then de-stained in 100% methanol. Blue spots were counted using a dissecting microscope at 5×-20× magnifications.

To quantitate the avirulence activity of Avr1b-1 constructs, DNA carrying the constructs (1.7 ug per shot) was co-bombarded into soybean leaves along with DNA carrying a β-glucuronidase (GUS) reporter gene (1.7 ug per shot). Avirulence activity was measured as the reduction in the number of blue-staining GUS-positive spots in leaves carrying Rps1b compared to leaves lacking Rps1b. The double-barreled device was used to deliver a parallel control shot in every case that contained GUS DNA plus empty vector DNA. For each pair of shots, the logarithm of the ratio of the blue spots with Avr1b to that with the empty vector control was calculated. Each assay consisted of eight pairs of shots and was conducted at least twice. The log-ratios from all the Rps1b leaves were then compared to those from the non-Rps1b leaves using the Wilcoxon rank sum test.

To quantitate suppression of BAX-mediated cell death, two assays were used, an indirect assay and a direct assay. For the indirect assay, Avr1b-1 DNA (1.7 ug/shot) was mixed with Bax DNA (pUCBax; 0.83 ug/shot) and GUS DNA (1.7 ug/shot) and bombarded into soybean leaves lacking Rps1b. The control shot in the second barrel was empty vector (pUC19; 2.53 ug/shot) plus GUS DNA (1.7 ug/shot). The log-ratios for these shots were then compared to the log-ratios obtained when Avr1b-1 DNA was replaced by empty vector DNA; 14-16 pairs of shots were carried out for each comparison, and the results were evaluated using the Wilcoxon rank sum test. The indirect assay had the advantage that the level of PCD triggered by BAX could be monitored in every shot. For the direct assay, Avr1b+BAX+GUS was compared directly with empty vector+BAX+GUS in the second barrel. The log-ratios obtained were then tested for significance using the Wilcoxon signed-ranks test. Again 14-16 pairs of shots were carried out. The direct assay had the advantage that the activity of Avr1b-1 could be compared to a control (or another Avr1b-1 construct) directly on the same leaves. Other effectors or mutants were tested by replacing the Avr1b-1 DNA with the relevant DNA.

### Example 4

### Use of the DoubleShot Gene Gun (the Current Invention) to Identify Pathogen Virulence Proteins

The usefulness of the DoubleShot device is its ability to measure the difference between two similar treatments. Most typically the Double Shot measures killing of plant cells as the result of the action of a gene of interest. The DoubleShot introduces genes under the control of a plant promoter (pNOS or p35S) into plant cells by bombardment with tungsten particles. A beta-galacturonidase reporter gene (GUS) is used to measured cell transformation (by DNA uptake). When the GUS gene is present in living cells, the cells stain blue with the dye X-gluc. Killing is measured by counting the number of surviving transformed cells with and without the cell killing gene.

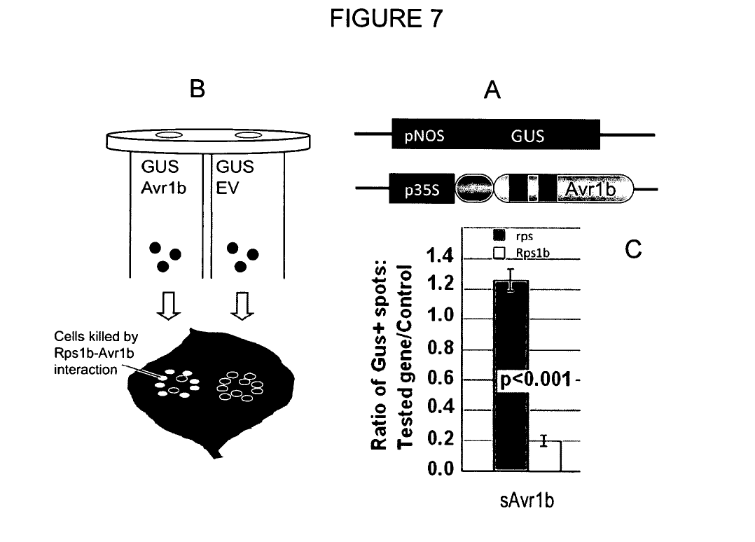

In the use illustrated in FIG. 7, plant cells are killed as a result of the action of a pathogen virulence gene, Avr1b from the soybean pathogen Phytophthora sojae, which kills soybean cells which contain the gene Rps1b (Dou et al, 2008a,b). In this assay, 16 double shots were assayed on leaves with no Rps1b gene (rps) and 16 on leaves with Rps1b present. One barrel contained GUS and control (EV) DNA and one contained GUS and Avr1b DNA. The ratio of spots with and without Avr1b was averaged for all 16 shots and statistically compared between rps and Rps1b. DNA constructions are described in detail in the methods. This assay has also been used to identify other virulence proteins from Phytophthora sojae, namely Avr4/6, Avr1k, Avr1a and Avr3a (Table 8) (data not shown). The DNA sequence for each virulence gene (sometimes known as an “avirulence” or “effector” gene, was fused to the CaMV 35S promoter and NOS terminator as described in the methods. The portion of each gene encoding the secretory leader was replaced with an ATG methionine initiation codon. Where available GenBank accession number for the genes are given. Otherwise the sequences are noted below. Soybean cultivars used were Williams (no Rps gene), L75-6141 (Rps1a), L77-1870 (Rps1b), Williams 82 (Rps1k), L83-570 (Rps3a), L85-2352 (Rps4), L89-1581 (Rps6). Ratios (±standard errors) of numbers of blue-stained cells expressing β-glucuronidase were obtained in the presence of the virulence gene DNA compared to the presence of control plasmid vector DNA. p value indicates significance of difference between ratios obtained with and without the Rps gene present. A value less that 0.05 indicates significant cell killing. The amino acid sequence of Avr1a is: FSAATDADQATVSKLAAAEFDTLVDVLTTESKRSLRATVDDGEE RYKQFKIEALKKGKWTDIFNKWKGNELSPAEVQNKLKNKKLSDDLKDAIFRNYKDW (SEQ ID NO:35). The amino acid sequence of Avr3a is: LSTTNANQAKIIKGTSPGGHSPRL LRAYQPDDEGDSPEDRTLSKAQVTKILNKLGKDVTWDHVMRNPALFQRYQKKANKIIE KQKAAAKNA (SEQ ID NO:36).

TABLE 8

| | | Bombardment results | | |
|---|---|---|---|---|
| P. sojae Virulence | Soybean | Control | Rps | |
| gene | Rps gene | (no rps gene) | gene present | p value |
| Avr1b AAM20936.1 | Rps1b | 1.26 ± 0.07 | 0.28 ± 0.03 | <0.001 |
| Avr1b | Rps1k | 1.25 ± 0.03 | 0.26 ± 0.04 | <0.001 |
| Avr1kEU282487 | Rps1k | 0.99 ± 0.03 | 0.64 ± 0.04 | <0.001 |
| Avr4/6ABS50087 | Rps4 | 1.25 ± 0.05 | 0.4 ± 0.06 | <0.001 |
| Avr4/6 | Rps6 | 1.21 ± 0.07 | 0.55 ± 0.1 | <0.001 |
| Avr1a | Rps1a | 0.99 ± 0.03 | 0.12 ± 0.05 | <0.001 |
| Avr3a | Rps3a | 0.98 ± 0.04 | 0.14 ± 0.03 | <0.001 |

### Example 5

### Use of the DoubleShot Gene Gun to Identify Toxins and Produce Dose-Response Curves

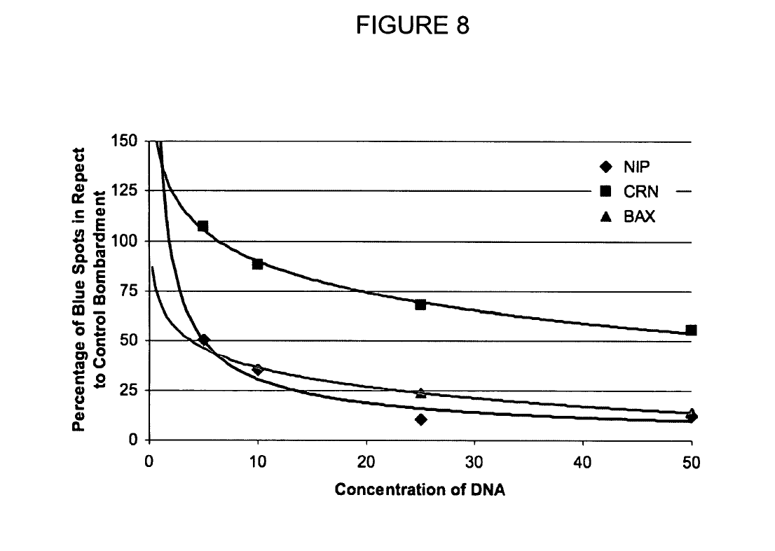

This application is similar to the detection of virulence genes except that the cell killing may be caused by a gene from any organism that causes cell death. Furthermore, the cell killing does not require any particular soybean gene (such as an Rps gene to be present). FIG. 8 shows the cell killing caused by three toxins, NIP and CRN from P. sojae (unpublished) and BAX from mouse (Dou et al., 2008a). The figure also illustrates the ability of the double shot to produce precise dose-response curves, which is not possible with any other kind of bombardment device. NIP indicates the P. sojae toxin encoded by the NPP1 gene (AAK01636.1). CRN indicates the P. sojae protein encoded by mRNA sequences similar to crinkling and necrosis inducing proteins of P. infestans, the sequence of which is: AGQSAGDLKDAIKAKNPATITCDAKDLQLSLAKTADGAWLPDDDQAA LDLEDGKVHEDIQALIDGEKMKATWTIEDVLTANNMTKRKGRAPKSRQIHVLVVVPEG AFGSASETSKMDQLVEKVDKMYEQTVLGKRKYVHSEVTSTQGRQLLNDLDIRVEFVRT VPFDAGEGSSVDPYEWKRVIIENGEEVVLTEEQQRKRYRRYVEHNIGAVLKEKQLCVIG VERGTNILTVKVPGREIELAGRTDLLILSDLVAMRPTEVQYLPGVKMLIEVKRDVKASN DFQALSELIALDLLVDDPVMALLTDLKGEWIFFWVAEKINSSARIHKAAINKPGEAFEVI RALLVQPPTAPADTDTTEIKLPCFQSPVKRLKLRKALPPIGEGGDNGGIRESIERYYDIAS MLGPDIEMARAVARQVTRSIPTFSYFS (SEQ ID NO:37). BAX indicates the mouse BAX protein (Q07813).

### Example 6

### Use of the DoubleShot Gene Gun to Identify Proteins that can Protect Against Toxins

In this application, a gene encoding a potential suppressor of cell death is introduced together with a toxin gene (BAX) (Dou et al., 2008a). Two assays are illustrated, the indirect and the direct assay. For the indirect assay, suppressor DNA (e.g. Avr1b) was mixed with Bax DNA and GUS DNA and bombarded into soybean leaves. The control shot in the second barrel was empty vector plus GUS DNA. The ratios for these shots were then compared to the ratios obtained when Avr1b DNA was replaced by empty vector DNA. The indirect assay had the advantage that the level of cell killing triggered by BAX could be monitored in every shot. For the direct assay, Avr1b+BAX+GUS was compared directly with empty vector+BAX+GUS in the second barrel. The direct assay had the advantage that the activity of the suppressor could be compared to a control directly on the same leaves. Other effectors were tested by replacing the Avr1b DNA with the relevant DNA (Dou et al., 2008a). Soybean bombardment assays of suppression of BAX mediated toxicity by four pathogen proteins from two different pathogen species is shown below in Table 9. The ratio from the indirect assay is calculated as (BAX+gene/EV ratio)/(BAX/EV ratio). The p value was calculated for the comparison using the Wilcoxon rank sum test. The p value for the direct assay was calculated using the Wilcoxon signed rank test. The average ratio and standard error was taken from 14-16 pairs of shots. The Anti-PCD Activity of each gene was determined by whether the ratios for both direct and indirect assays were statistically significantly greater than 1.0. The species from which the suppressor gene was obtained were P. sojae (Ps) and the downy mildew pathogen Hyaloperonospora parasitica (Hp). In this example, three different DNA molecules are introduced in one or both barrels, showing the versatility of the device of the invention.

TABLE 9

| | Indirect assay | | | | Direct assay | |
|---|---|---|---|---|---|---|
| | (BAX + EV)/ | (Gene + BAX)/ | | | (BAX + Gene)/ | |
| Suppressor Gene | EV | EV | Ratio | p value | (BAX + EV) | p value |
| Ps Avr1b (AAM20936.1) | 0.12 ± 0.02 | 0.34 ± 0.04 | 2.84 | p < 0.001 | 3.36 ± 0.66 | p < 0.001 |
| PsAvh163 (EU282485) | 0.10 ± 0.02 | 0.30 ± 0.13 | 3.00 | p < 0.001 | 1.57 ± 0.26 | p < 0.001 |
| PsAvh331 (EU282487) | 0.10 ± 0.01 | 0.60 ± 0.03 | 6.93 | p < 0.001 | 6.17 ± 0.66 | p < 0.001 |
| HpRXL96 (EU282490) | 0.12 ± 0.03 | 0.39 ± 0.06 | 3.25 | p < 0.001 | 3.62 ± 0.74 | p < 0.001 |

### Example 7

### Use of the DoubleShot Device to Determine the Cellular Site of Action of Avirulence Protein or Toxin

In this example, an amino sequence that directs an expressed protein to a particular cellular compartment is attached to the protein and the protein is then assayed using the DoubleShot bombardment assay. This assay would not be possible without the unique precision offered by the device of the invention. In the example shown in Table 10, sequences that direct Avr1b into the nucleus (NLS) or out of the nucleus (NES1) were attached to Avr1b, then its ability to suppress the action of the toxin BAX and its ability to kill soybean cells when Rps1b is present were assayed. The cellular targeting results show that when Avr1b is restricted to the nucleus, it can kill soybean cells containing Rps1b but can no longer suppress killing by BAX. Conversely, when Avr1b is excluded from the nucleus, it can suppress killing by BAX but can no longer kill soybean cells containing Rps1b. The results indicate that Avr1b has two sites of action in the cell, one in the nucleus and one outside of it. Sequences were attached to the C terminus of Avr1b. NLS (nuclear localization signal) sequence was GAPKKKRKVK(SEQ ID NO:38). NES1 (functional nuclear export signal) sequence was NELALKLAGLDINK (SEQ ID NO:39). NES2 (non-functional nuclear export signal) sequence was NELALKAAGADANK (SEQ ID NO:40). The indirect assay of Avr1b cell killing was performed as described for FIG. 7 and the methods above. A number significantly less than 1 indicates Avr1b is functional in killing soybean cells. p indicates statistical significance (p should be less than 0.05 for significance). The indirect assay of Avr1b suppression of BAX cell killing was performed as described for Table 10 and the methods above. A number significantly greater than 1 indicates Avr1b is functional in suppressing BAX killing of soybean cells. p indicates statistical significance (p should be less than 0.05 for significance).

TABLE 10

| Targeting sequence | Killing in presence of | Suppression of cell killing |
|---|---|---|
| attached to Avr1b | Rps1b | caused by BAX |
| NLS | 0.065; p < 0.001 | 0.48 ± 0.1; p > 0.1 |
| NES1 | 1.03; p > 0.1 | 2.75 ± 0.4; p < 0.001 |
| NES2 | 0.14; p < 0.001 | 3.3 ± 0.3; p < 0.001 |
| None | 0.22 ± 0.07; p < 0.001 | 2.8 ± 0.3; p < 0.001 |

### Example 8

### Use of the DoubleShot Device to Measure the Ability of a Protein to Enter Plant Cells

In this assay, the N-terminus of Avr1b is mutated or deleted, preventing it from entering soybean cells (Dou et al., 2008b). The N-terminus of another protein is attached to Avr1b together with a secretory leader that directs the protein out of the cell when it is produced in plant cells. If the N-terminus of a foreign protein can transport back into the soybean cells, then cell-killing is observed in soybean cells containing the Rps1b gene.

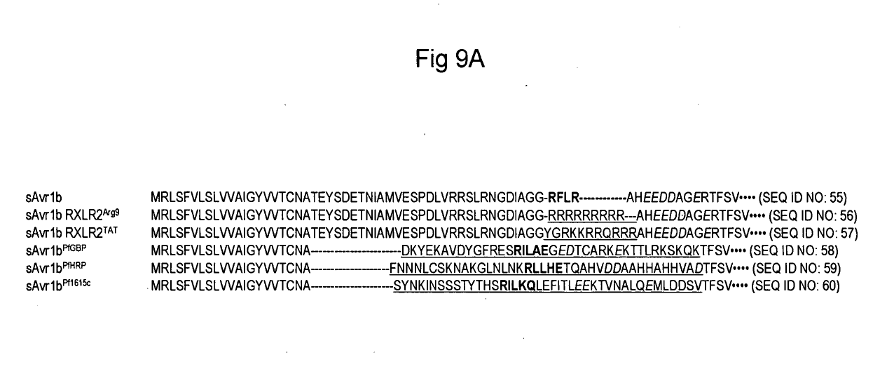

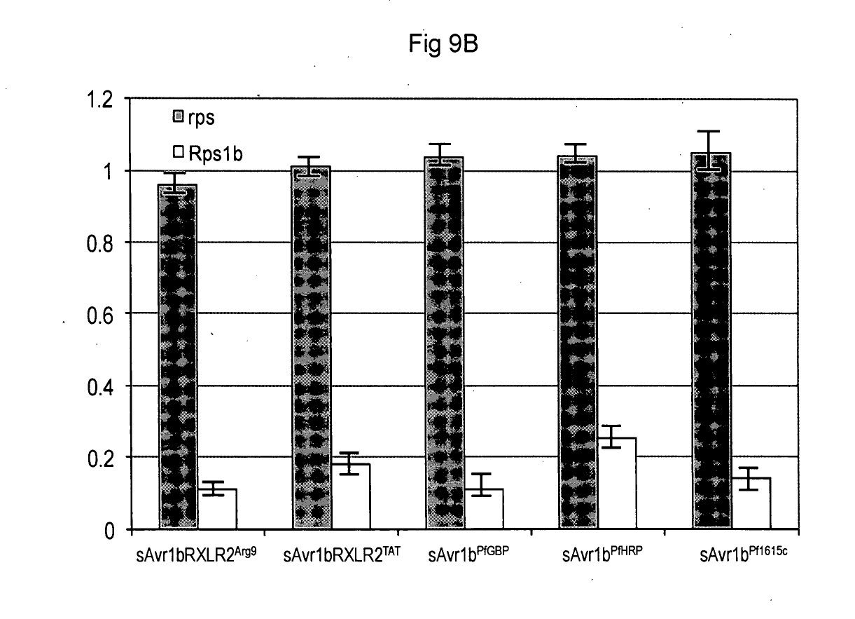

FIG. 9 shows that sequences from the HIV TAT protein and from the N-terminus of three proteins from the malaria parasite Plasmodium can transport Avr1b back into plant cells (Dou et al., 2008b). An artificial sequence (Arg₉) can also do this (Dou et al., 2008b).

FIG. 9 depicts the functional replacement of Avr1b transport signal with protein transduction motifs and Plasmodium host targeting signals. Panel A shows the sequences of modified Avr1b proteins. Arg9 refers to a synthetic amino acid sequence of nine consecutive arginines; TAT refers to the HIV TAT protein (BAF32556.1); PfGBP, PfHRP and Pfl615c refer to the Plasmodium PfGBP-130 (AAN35357.1), PfHRPII (AAX28692.1) and PfPFE1615c (XP_{—}001351874.1) proteins (Bhattacharjee et al., 2006). All non-native Avr1b sequences are underlined, Avr1b RXLR2 and Plasmodium RXLX^{E}/_{Q} motifs are in bold, and acidic residues in the dEER region are in italics. The Avr1b secretory leader was used in all constructs. Panel B shows the ratio of blue spots in the presence of Avr1b-1 compared to the control, assayed as described in FIG. 9. Constructs are as in Panel A. Averages and standard errors are from 8 pairs of shots.

### Example 9

### Expression of Proteins from Different Species Using the DoubleShot Device

Table 11 contains a summary of the expression of proteins from different species using the DoubleShot device of the invention. As can be seen, the device has been tested using nucleic acids from a wide variety of species ranging from parasites to plants and animals.

TABLE 11

| | | | |
|---|---|---|---|
| Source organism | Protein | Function | Data |

| | | | |
|---|---|---|---|
| E. coli | GUS | Enzyme activity | FIG. 7 |
| Phytophthora sojae | Avr1b | virulence protein | Tables 8 and 9 |
| | | and suppressor of | |
| | | BAX toxin | |
| Phytophthora sojae | Avr4/6 | virulence protein | Table 8 |
| Phytophthora sojae | Avh331 | suppressor of BAX | Table 9 |
| | | toxin | |
| Phytophthora sojae | Avr1a | virulence protein | Table 8 |
| Phytophthora sojae | Avr3a | virulence protein | Table 8 |
| Phytophthora sojae | CRN | toxin | FIG. 8 |
| Phytophthora sojae | NIP | toxin | FIG. 8 |
| Hyaloperonospora | Hp RxL96 | suppressor of BAX | Table 9 |
| parasitica | | toxin | |
| mouse | BAX | toxin | FIG. 8 |
| Wheat stripe | Ps87 Nt^{a} | Transport of Avr1b | 0.34 ± 0.07; |
| rust fungus | | protein | p < 0.001^{b} |
| Puccinia striiformis | | | |
| (Pst) | | | |
| Rice blast fungus | Ps87-like Nt^{a} | Transport of Avr1b | 0.18 ± 0.02; |
| Magnaporthe grisea | | protein | p < 0.001^{b} |
| (Mg) | | | |
| Flax rust fungus | AvrL567 Nt^{a} | Transport of Avr1b | 0.30 ± 0.09; |
| Melampsora lini (Ml) | | protein | p < 0.001^{b} |
| Flax rust fungus | AvrM Nt^{a} | Transport of Avr1b | 0.34 ± 0.05; |
| Melampsora lini | | protein | p < 0.001^{b} |
| Flax rust fungus | AvrP123 Nt^{a} | Transport of Avr1b | 0.22 ± 0.03; |
| Melampsora lini | | protein | p < 0.001^{b} |
| Malaria parasite | PfGBP-130 Nt^{a} | Transport of Avr1b | FIG. 9 |
| Plasmodium falciparum | | protein | |
| (Pf) | | | |
| Malaria parasite | PfHRPII Nt^{a} | Transport of Avr1b | FIG. 9 |
| Plasmodium falciparum | | protein | |
| Malaria parasite | PfPFE1615c Nt^{a} | Transport of Avr1b | FIG. 9 |
| Plasmodium falciparum | | protein | |
| Virus HIV | TAT Nt^{a} | Transport of Avr1b | FIG. 9 |
| | | protein | |

^{a}Nt = N-terminus of the protein fused to amino acids X to X of Avr1b. N-terminal sequences are: Pst Ps87
TLLNVTRRLQNDGKPPDYCVDKWDEMMKERNKRLTGKPRGQCVDEI; (SEQ ID NO:41) Mg Ps87-like (from XP_362169)
GISTVRWYTNGGKRPRRSIDQWDRQMMDRDRRLTGFLRGQSDNPIA; (SEQ ID NO:42) Ml AvrL567 (from AAS66948.1)
MEHVPAELTRVSEGYTRFYRSPTASVILSGLVKVKWDNEQMTM; (SEQ ID NO:43) Ml AvrM (from ABB96264.1):
HPMNSAKLAEEVKDGVNQPEFDRGFLRPFGAKMKFLKPDQVQKLSTDDLITYMAEKDKN; (SEQ ID NO:44) Ml AvrP123 (from ABB96267.1)
QYVVDPGFGEIECMCGQIARLTQRPFDVECEAT. (SEQ ID NO:45) Plasmodium and HIV sequences are described in FIG 9.
^{b}Assay of cell killing in the presence of Rps1b as described in FIG. 7 and the Methods.

### Example 10

### Use of the DoubleShot Device to Accurately Measure Protein Function in Detailed Structure-Function Assays

Case 1. Mutational Analysis of the Protein Transport Motif RXLR in Avr1b.

In this example, the function of RXLR2 mutants of Avr1b was assayed by particle bombardment using the DoubleShot device. The RXLR2 motif of Avr1b which is required for Avr1b to enter soybean cells was mutated from its normal sequence, RFLR, to the sequences indicated in column 1 of Table 12. The results indicate that RXLR2 will tolerate very few changes.

TABLE 12

| | Ratio of | | | | |
|---|---|---|---|---|---|
| RXLR2 | GUS-positive spots^{b} | | | | |
| sequence^{a} | rps | Rps1b | ablation^{c} | p value^{d} | Activity |
| RFLR | 1.26 ± 0.07 | 0.28 ± 0.03 | 0.78 | <0.001 | Yes |
| AAAA | 0.93 ± 0.04 | 0.96 ± 0.05 | 0 | >0.1 | No |
| KFLR | 1.04 ± 0.04 | 0.70 ± 0.04 | 0.33^{e} | <0.001 | Partial |
| QFLR | 0.95 ± 0.03 | 0.99 ± 0.03 | 0 | >0.1 | No |
| FRLR | 1.00 ± 0.04 | 0.98 ± 0.05 | 0 | >0.1 | No |
| RFLQ | 0.98 ± 0.07 | 0.41 ± 0.08 | 0.58^{e} | <0.001 | Partial |
| QFLQ | 1.03 ± 0.05 | 1.05 ± 0.05 | 0 | >0.1 | No |
| RFAR | 0.94 ± 0.03 | 0.91 ± 0.05 | 0 | >0.1 | No |
| RFVR | 0.95 ± 0.05 | 1.03 ± 0.07 | 0 | >0.1 | No |
| RFRL | 1.02 ± 0.04 | 0.96 ± 0.04 | 0 | >0.1 | No |

^{a}Amino acid sequence of RXLR2 in wild-type and mutants. RFLR is the wild-type. Altered residues are underlined. In the following first 40 amino acids of Avr1b, the RXLR2 sequence is underlined: TEYSDETNIAMVESPDLVRRSLRNGDIAGGRFLRAHEEDD (SEQ ID NO: 46).
^{b}Ratio of blue spots in the presence of various RXLR2 mutants of Avr1b-1, compared to the control empty vector when bombarded onto leaves from rps plants (Williams) or Rps1b plants (L77-1863). Averages and standard errors are from 16 pairs of shots.
^{c}Ablation calculated as 1 − (Rps1b ratio)/(rps ratio) for ratios significantly different between rps and Rps1b.
^{d}p values comparing results from rps and Rps1b cultivars were calculated using the Wilcoxon rank sum test.
^{e}Ablations for KFLR and RFLQ were significantly different than wildtype (RFLR) with p < 0.001.

Case 2. Mutational Analysis of the Toxin Suppression Motif W-Y in Avr1b

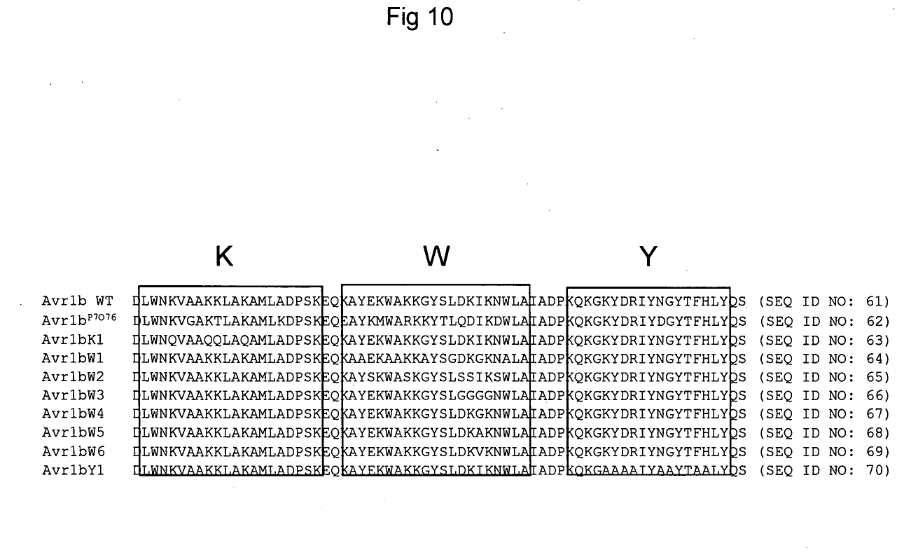

In this example (from Dou et al., 2008a), the effects of Avr1b mutations on PCD suppression were monitored. Mutations were introduced in conserved amino acid residues in the C-terminus and then the mutant genes were assayed using the DoubleShot device. The mutants of Avr1b are shown in FIG. 10. P7076 is a natural sequence variant of Avr1b (AAM20939.1). The results show that many of the conserved amino acids in the W and Y motifs are required for Avr1b to suppress BAX killing, but the K motif is not required (Table 13).

TABLE 13

| | PCD suppression^{b} | | | |
|---|---|---|---|---|
| | Direct | | Indirect | |
| Mutant^{a} | Ratio to control | p value | Ratio to control | p value |
| Avr1b⁺ | 3.36 | <0.001 | 2.84 | <0.001 |
| Avr1b^{P7076} | 0.74 | >0.5 | 0.74 | >0.1 |
| Avr1bK1 | 2.41^{c} | <0.001 | 4.87^{c} | <0.001 |
| Avr1bW1 | 0.99 | >0.4 | 0.68 | >0.1 |
| Avr1bW2 | 3.48 | <0.001 | 3.81 | <0.001 |
| Avr1bW3 | 0.85 | >0.5 | 0.67 | >0.1 |
| Avr1bW4 | 1.03 | >0.2 | 0.82 | >0.1 |
| Avr1bW5 | 0.93 | >0.5 | 1.06 | >0.1 |
| Avr1bW6 | 3.12 | <0.001 | 3.46 | <0.001 |
| Avr1bY1 | 1.80^{d} | <0.01 | 1.55^{e} | <0.05 |

^{a}Sequences of Avr1b mutant proteins given in FIG. 10.
^{b}Suppression of BAX-mediated cell killing was measured using both the direct and indirect assays as described in the Methods section and in the legend of Table 10. Significant deviation above 1.0 and p value less than 0.05 indicates activity in suppression.
^{c}not significantly different than wild type Avr1b, p > 0.1
^{d}significantly different than wild type Avr1b, p < 0.025
^{e}significantly different than wild type Avr1b, p < 0.001

It will be apparent to those skilled in the art that various modifications and variations can be made in the practice of the method of the present invention and in construction of the device of the invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

### REFERENCES

Allen, R. L. et al., Science 306, 1957 (2004).
Armstrong, M. R. et al., Proc Natl Acad Sci USA 102, 7766 (2005).
Bhattacharjee, S. et al., PLoS Pathol 2, e50 (2006).
Birch, P. R. et al., Trends Microbiol 14, 8 (2006).
Bos, J. I. B. et al., Plant Journal 48, 165 (2006).
Buzzell, R. I. et al., Soybean Genet. Newsl. 14, 79 (1987).
Chang, M. et al., New Phytol 174, 46 (2007).
Dou, D. et al., Plant Cell 20(4), 1118-1133 (2008).
Dou, D. et al., Plant Cell, in press.
Erwin, D. C. and Ribiero, O. K., Phytophthora Diseases Worldwide (APS Press, St. Paul, Minn., 1996).
Förster, H. et al., Mol. Plant-Microbe Interact. 7, 780 (1994).
Hiller, N. L. et al., Science 306, 1934 (2004).
Jiang, R. H. Y. et al Proc. Natl. Acad. Sci. USA 105, 4874-4879 (2008).
Joliot, A., Sci STKE 2005, pe54 (2005).
Judelson, H S et al., Mol. Plant-Microbe Interact. 6, 602-607. (1991).
Kaplan, I. M. et al., J Control Release 102, 247 (2005).
Klein, T. M., et al., Nature 327: 70-73 (1987).
Klein, T. M. et al., Nature Biotechnology 10: 286-291 (1992).
Marti, M. et al., Science 306, 1930 (2004).
Mindrinos, M. et al., Cell 78, 1089 (1994).
Qutob, D. et al., Plant J 32, 361 (2002).
Rehmany, A. P. et al., Plant Cell 17, 1839 (2005).
Sambrook, J. and Russell, D W. Molecular Cloning. Cold Spring Harbor, N.Y.: Cold Spring Harbor Laboratory Press. (2001)
Sanford, J. C. et al., Technique 3: 3-16 (1991).
Schledzewski, K. and Mendel, R. R., Transgenic Research 3: 249-255 (1994).
Shan, W. et al., Mol. Plant. Microbe Interact 17, 394 (2004).
Snyder, E. L. and Dowdy, S. F., Pharm Res 21, 389 (2004).
Staskawicz, B. J. et al., Science 292, 2285 (2001).
Tomasinsig, L. et al., J Biol Chem 281, 383 (2006).
Tyler, B. M., Annual Rev. Phytopath. 40, 137 (2002).
Tyler, B. M. et al., Science 313, 1261 (2006).
Wade, M. and Albersheim, P., Proc Natl Acad Sci US A.
Yoon, H. S. et al., Proc Natl Acad Sci USA 99, 15507 (2002).

1. A device for introducing one or more payloads into a biological cell of a biological tissue, said device comprising:
an apparatus capable of ejecting said payload into said cell, and
an attachment for focusing said payload on a specific location of said tissue,
wherein said attachment comprises at least two distinct chambers for transmission of said payload(s) from said apparatus to said cell, and
wherein said attachment is connected to said apparatus such that an aperture or opening in said apparatus for ejection of said payload is aligned with said chambers such that ejection of said payload permits the payload to traverse the chambers and contact said cell.

2. The device of claim 1, wherein the payload is a biological or chemical material.

3. The device of claim 1, wherein the attachment for focusing the payload comprises two hollow cylinders or tubes.

4. The device of claim 1, wherein the attachment is attached to the apparatus by way of a retaining spring or a screw thread.

5. The device of claim 1, wherein the device is comprised of a metal or plastic, or a combination of both.

6. An attachment for a biolistic transformation apparatus, said attachment comprising:
a connector for connecting the attachment to the apparatus, and
at least two chambers for delivery of a payload from the apparatus to a target.

7. The attachment of claim 6, comprising two chambers.

8. The attachment of claim 6, wherein the connector comprises a spring clip or screw thread.

9. A method of introducing a payload into a biological cell, said method comprising:
contacting the cell with the payload with sufficient force to cause the payload to enter the interior of the cell,
wherein the force propelling the payload is provided by the device of claim 1.

10. The method of claim 9, wherein the contacting does not kill the cell.

11. The method of claim 9, wherein the payload is a biological or chemical material.

12. The method of claim 9, wherein the payload is nucleic acid.

13. The method of claim 11, wherein the nucleic acid is DNA.

14. The method of claim 11, wherein the cell is rendered recombinant or transformed.

15. The method of claim 14, further comprising exposing the recombinant or transformed cell to conditions that allow the cell to live and, optionally, reproduce.

16. A method of introducing a nucleic acid into a biological cell, said method comprising:
contacting said cell with a nucleic acid emitted from an apparatus with sufficient force to cause the nucleic acid to enter the cell,
wherein said apparatus comprises a biolistic transformation apparatus comprising an attachment comprising two or more chambers that focus the nucleic acid onto specific cells of a tissue.

17. The method of claim 16, wherein the method produces a transformed plant cell.

18. The method of claim 17, wherein the plant cell is a transgenic plant cell that stably maintains the introduced nucleic acid.
